SEQUENCE LISTING (8 sequences)

SEQ ID NO: 1 - DNA, 213 bp, synthetic construct
  misc_feature 1..213: note = Synthetic: MIP-3 alpha
  source 1..213: mol_type = other DNA
gccgctagcaacttcgactgctgtctgggatacacagatagaatcctgcacccaaagttcatcgtgggctttaccagacagctggccaacgagggatgcgacatcaacgctatcatctttcacaccaagaagaagctgagcgtgtgcgccaaccccaagcagacatgggtgaagtacatcgtgcggctgctgagcaagaaggtgaagaacatg

SEQ ID NO: 2 - DNA, 57 bp, synthetic construct
  misc_feature 1..57: note = Synthetic: Linker
  source 1..57: mol_type = other DNA
ggaccaggacctggaccaggaccaggacctcaggcgccgaagagtctcgaggctagc

SEQ ID NO: 3 - DNA, 2211 bp, synthetic construct
  misc_feature 1..2211: note = Synthetic: Mouse codon optimized RelMtb
  source 1..2211: mol_type = other DNA
accgcccagaggtctaccacaaaccctgtgctggagccactggtggcagtccacagggagatctaccccaaggccgatctgagcatcctgcagagggcatatgaggtggcagaccagaggcacgccagccagctgcgccagtccggcgatccttacatcacacacccactggccgtggccaatatcctggccgagctgggcctggacaccacaaccctggtggccgccctgctgcacgacaccgtggaggatacaggctataccctggaggccctgacagaggagttcggagaggaagtgggacacctggtggacggagtgaccaagctggatagggtggtgctgggctccgccgcagagggagagacaatcagaaagatgatcacagcaatggccagggaccccagggtgctggtcatcaaggtggccgaccggctgcacaacatgaggaccatgagattcctgccacctgagaagcaggcaaggaaggccagggagacactggaagtgatcgcaccactggcccacaggctgggaatggcctctgtgaagtgggagctggaggacctgagctttgccatcctgcaccctaagaagtacgaggagatcgtgcggctggtggcaggaagggcaccaagcagagatacctatctggccaaggtgcgcgccgagatcgtgaatacactgaccgcctctaagatcaaggccacagtggagggcaggcccaagcactactggagcatctatcagaagatgatcgtgaagggcagagacttcgacgatatccacgatctggtgggcgtgagaatcctgtgcgacgagatccgcgattgttacgcagcagtgggagtggtgcacagcctgtggcagccaatggcaggccggtttaaggactatatcgcccagccccgctacggcgtgtatcagtccctgcacacaaccgtggtgggaccagagggcaagcctctggaggtgcagatccggacccgcgatatgcacaggacagcagagtacggaatcgcagcacactggaggtataaggaggccaagggcagaaacggcgtgctgcaccctcacgcagcagcagagatcgacgatatggcctggatgaggcagctgctggactggcagagggaggcagccgatcccggagagttcctggagtctctgcgctacgacctggccgtgcaggagatcttcgtgtttacccctaagggcgacgtgatcacactgcccaccggcagcacacctgtggattttgcctatgcagtgcacacagaagtgggacacaggtgcatcggagcccgggtgaacggccgcctggtggccctggagcgcaagctggagaatggcgaggtggtggaggtgtttaccagcaaggcaccaaacgcaggaccctccagagactggcagcagttcgtggtgtccccaagggccaagaccaagatcagacagtggtttgccaaggagaggagagaggaggccctggagacaggcaaggatgccatggcccgggaggtgcggaggggaggcctgcccctgcagcgcctggtgaatggagagtctatggcagcagtggccagggagctgcactacgcagacgtgagcgccctgtataccgcaatcggagagggacacgtgtccgccaagcacgtggtgcagagactgctggccgagctgggaggaatcgatcaggccgaggaggagctggccgagaggtctaccccagccacaatgcccaggaggcccagatctaccgacgatgtgggcgtgagcgtgccaggagcaccaggcgtgctgaccaagctggccaagtgctgtacaccagtgcccggcgacgtgatcatgggattcgtgacaaggggcggaggcgtgtccgtgcacagaaccgattgtacaaacgcagcctctctgcagcagcaggcagagaggatcatcgaggtgctgtgggccccttccccaagctccgtgtttctggtggccatccaggtggaggccctggacaggcacagactgctgtctgatgtgaccagagccctggccgacgagaaagtgaatatcctgtctgccagcgtgacaacctccggcgacagggtggccatcagcaggttcaccttcgagatgggcgatcctaagcacctgggccacctgctgaacgccgtgaggaatgtggagggcgtgtacgacgtgtatagagtgacctccgccgcc

SEQ ID NO: 4 - DNA, 2370 bp, synthetic construct
  misc_feature 1..2370: note = Synthetic: Non-codon optimized RelMtb
  source 1..2370: mol_type = other DNA
gtggccgaggaccagctcacggcgcaagcggttgcaccgcccacggaggcttctgcggctctcgagcccgctctcgagacgcccgagtcgccggtcgagactcttaagaccagcatcagcgcgtcgcgtcgggtgcgggcccgattggcccggcggatgaccgcccagcgcagcaccaccaatccggtgctcgagccgttggtggcggtgcaccgggagatctatcccaaggccgacctgtcgatcttgcagcgagcctacgaggtcgctgaccaaaggcatgccagccagttgcggcagtccggtgatccctacatcacccacccgttggccgttgccaacattctggccgagttgggcatggacaccaccactttggtggccgcgctgctgcacgacaccgtcgaggacaccggttacaccctggaggcgttgaccgaggaattcggcgaagaggtgggccatctcgtcgacggggtgaccaagctggatcgggtggtgttgggcagcgccgccgaaggcgagactattcgcaagatgatcaccgcgatggcccgcgatccgcgggtgctggtgataaaggtggctgaccggttacacaacatgcgcaccatgcgcttcttgccgccggagaagcaggcccgcaaggcccgtgagacgttggaagtcattgcacccctggcgcatcggctgggcatggccagcgtcaagtgggagttggaggacctgtccttcgcgatcctgcatcccaagaagtacgaggagatcgtccggctggtcgccggtcgcgcgccgtcccgggacacctacctggccaaggtgcgtgccgaaatcgtcaacacgctgaccgcgtcgaagatcaaggcgacggtggagggccgccccaagcactattggtcgatctaccagaagatgatcgttaagggccgcgacttcgacgacatccacgacctggtcggtgtgcgcatcctgtgcgacgaaatccgggactgctacgcggctgtcggcgtagtgcattcgctatggcagccgatggcgggtcggttcaaggactacatcgcccagcccagatacggtgtgtaccagtcactgcacaccactgtggtcgggcctgagggcaagccgctggaagtgcagatccgtacccgcgacatgcaccgcaccgccgaatacggcatcgccgcgcattggcgctacaaagaagccaagggccgcaacggtgttcttcatccgcatgccgccgcggagatcgacgacatggcctggatgcgtcagctgctcgactggcaacgtgaggcggccgaccccggtgagttcttggaatcattgcgctacgaccttgcggtgcaagagattttcgtgtttacccccaagggcgacgtgatcacgctgccaaccggttcgacgccggtggacttcgcttacgcggtgcacacagaggtgggccaccgctgcatcggcgcccgagtgaacggccggttggtagcgctggaacgcaagctggaaaacggagaagttgtcgaggttttcacgtccaaggcgccgaacgccgggccgtcgcgggactggcagcagttcgtggtgtcgccgcgcgcaaagacgaagatccgccagtggttcgccaaggagcggcgtgaggaggcgttggagaccggtaaggatgcgatggcccgcgaggtgcgccgcggtggacttccgttgcagcgcttggtcaatggtgagtccatggcggcggtggcccgcgagctgcactacgcggacgtgtcagcactctataccgccatcggtgaggggcacgtgtcggcgaaacacgtcgtgcagcggttgttggccgagctcggcggtatcgaccaggcggaagaggaactcgccgagcggtccacgccggcgaccatgccgcggcgcccacgcagcaccgacgatgtcggggtctccgtccccggcgccccgggcgtgctgaccaagctggccaagtgctgcacgccggttccgggcgatgtgattatggggttcgtcacccgtggcggcggggtcagtgtgcaccgcaccgactgcaccaacgccgcatcgctgcagcagcaggccgagcgcatcatcgaggtgctatgggcgccgtcgccgtcgtcggtgtttctggtggcaatccaggtcgaggcactcgaccggcaccggctgctgtcggatgtgacgcgcgcactggccgacgagaaggtcaatatcctgtccgcgtcggtcaccacttcgggggaccgggtggcgatcagtcgattcaccttcgagatgggtgaccccaagcacctcgggcacctgctcaacgccgtccgcaacgtcgaaggtgtctacgacgtctaccgggtgacctcggccgcg

SEQ ID NO: 5 - protein, 71 aa, synthetic construct
  REGION 1..71: note = Synthetic: MIP-3 alpha
AASNFDCCLGYTDRILHPKFIVGFTRQLANEGCDINAIIFHTKKKLSVCANPKQTWVKYIVRLLSKKVKNM

SEQ ID NO: 6 - protein, 19 aa, synthetic construct
  REGION 1..19: note = Synthetic: Linker
GPGPGPGPGPQAPKSLEAS

SEQ ID NO: 7 - protein, 737 aa, synthetic construct
  REGION 1..737: note = Synthetic: Mouse codon optimized RelMtb
TAQRSTTNPVLEPLVAVHREIYPKADLSILQRAYEVADQRHASQLRQSGDPYITHPLAVANILAELGLDTTTLVAALLHDTVEDTGYTLEALTEEFGEEVGHLVDGVTKLDRVVLGSAAEGETIRKMITAMARDPRVLVIKVADRLHNMRTMRFLPPEKQARKARETLEVIAPLAHRLGMASVKWELEDLSFAILHPKKYEEIVRLVAGRAPSRDTYLAKVRAEIVNTLTASKIKATVEGRPKHYWSIYQKMIVKGRDFDDIHDLVGVRILCDEIRDCYAAVGVVHSLWQPMAGRFKDYIAQPRYGVYQSLHTTVVGPEGKPLEVQIRTRDMHRTAEYGIAAHWRYKEAKGRNGVLHPHAAAEIDDMAWMRQLLDWQREAADPGEFLESLRYDLAVQEIFVFTPKGDVITLPTGSTPVDFAYAVHTEVGHRCIGARVNGRLVALERKLENGEVVEVFTSKAPNAGPSRDWQQFVVSPRAKTKIRQWFAKERREEALETGKDAMAREVRRGGLPLQRLVNGESMAAVARELHYADVSALYTAIGEGHVSAKHVVQRLLAELGGIDQAEEELAERSTPATMPRRPRSTDDVGVSVPGAPGVLTKLAKCCTPVPGDVIMGFVTRGGGVSVHRTDCTNAASLQQQAERIIEVLWAPSPSSVFLVAIQVEALDRHRLLSDVTRALADEKVNILSASVTTSGDRVAISRFTFEMGDPKHLGHLLNAVRNVEGVYDVYRVTSAA

SEQ ID NO: 8 - protein, 790 aa, synthetic construct
  REGION 1..790: note = Synthetic: Non-codon optimized RelMtb
VAEDQLTAQAVAPPTEASAALEPALETPESPVETLKTSISASRRVRARLARRMTAQRSTTNPVLEPLVAVHREIYPKADLSILQRAYEVADQRHASQLRQSGDPYITHPLAVANILAELGMDTTTLVAALLHDTVEDTGYTLEALTEEFGEEVGHLVDGVTKLDRVVLGSAAEGETIRKMITAMARDPRVLVIKVADRLHNMRTMRFLPPEKQARKARETLEVIAPLAHRLGMASVKWELEDLSFAILHPKKYEEIVRLVAGRAPSRDTYLAKVRAEIVNTLTASKIKATVEGRPKHYWSIYQKMIVKGRDFDDIHDLVGVRILCDEIRDCYAAVGVVHSLWQPMAGRFKDYIAQPRYGVYQSLHTTVVGPEGKPLEVQIRTRDMHRTAEYGIAAHWRYKEAKGRNGVLHPHAAAEIDDMAWMRQLLDWQREAADPGEFLESLRYDLAVQEIFVFTPKGDVITLPTGSTPVDFAYAVHTEVGHRCIGARVNGRLVALERKLENGEVVEVFTSKAPNAGPSRDWQQFVVSPRAKTKIRQWFAKERREEALETGKDAMAREVRRGGLPLQRLVNGESMAAVARELHYADVSALYTAIGEGHVSAKHVVQRLLAELGGIDQAEEELAERSTPATMPRRPRSTDDVGVSVPGAPGVLTKLAKCCTPVPGDVIMGFVTRGGGVSVHRTDCTNAASLQQQAERIIEVLWAPSPSSVFLVAIQVEALDRHRLLSDVTRALADEKVNILSASVTTSGDRVAISRFTFEMGDPKHLGHLLNAVRNVEGVYDVYRVTSAA

(19) United States
(12) Patent Application Publication
(10) Pub. No.: US 2025/0222090 A1
(43) Pub. Date: Jul. 10, 2025

(54) TUBERCULOSIS VACCINES

(71) Applicant: THE JOHNS HOPKINS UNIVERSITY, Baltimore, MD (US)

(72) Inventors: Richard MARKHAM, Columbia, MD (US); Petros C. KARAKOUSIS, Towson, MD (US); Styliani KARANIKA, Baltimore, MD (US); James GORDY, Baltimore, MD (US); Yu-Min CHUANG, Orange, CT (US)

(21) Appl. No.: 18/853,636

(22) PCT Filed: Apr. 10, 2023
(86) PCT No.: PCT/US2023/065584

§ 371 Date: Oct. 2, 2024

Related U.S. Application Data
(60) Provisional application No. 63/329,655, filed on Apr. 11, 2022.

(51) Int. Cl. A61K 39/04; A61K 9/00; A61K 39/00; A61K 45/06; A61K 47/64; A61P 31/06; C12N 15/86

(52) U.S. Cl. CPC A61K 39/04; A61K 9/0043; A61K 45/06; A61K 47/646; A61P 31/06; C12N 15/86; A61K 2039/53; A61K 2039/543; A61K 2039/545; A61K 2039/55555; C12N 2800/22

(57) ABSTRACT

Provided herein are nucleic acid vaccine constructs comprising synthetic polynucleotides encoding a Mycobacterium tuberculosis (Mtb) RelA-SpoT homolog (RSH) protein, Rel_{Mtb}, or a functional portion, fragment, or variant thereof, conjugated to a macrophage inflammatory protein-3 alpha (MIP-3α) or other chemokine that binds to a chemokine receptor 6 (CCR6), or a functional portion, fragment, or variant thereof, or to an antibody, or antigen binding portion thereof, that binds to a CCR6. Methods for making the vaccine constructs and their use in prophylaxis and treatment of Mtb infections are also provided.

### CROSS-REFERENCE TO RELATED APPLICATIONS

This application is the national stage entry of International Patent Application No. PCT/US2023/065584, filed on Apr. 10, 2023, and published as WO 2023/201199 A9 on Oct. 19, 2023, which claims the benefit of U.S. Provisional Patent Application Ser. No. 63/329,655, filed on Apr. 11, 2022, which are hereby incorporated by reference in their entireties.

### STATEMENT REGARDING FEDERALLY SPONSORED RESEARCH OR DEVELOPMENT

This invention was made with government support under grants AI148710 and AI140860 awarded by the National Institutes of Health. The government has certain rights in the invention.

### REFERENCE TO ELECTRONIC SEQUENCE LISTING

The application contains a Sequence Listing which has been submitted electronically in XML format and is hereby incorporated by reference in its entirety. Said .XML copy, created on Apr. 5, 2023, is named “0184.0198-PCT.xml” and is 17,353 bytes in size. The sequence listing contained in this .XML file is part of the specification and is hereby incorporated by reference herein in its entirety.

### BACKGROUND OF THE INVENTION

Tuberculosis (TB) is a major cause of morbidity, and the second leading infectious killer after COVID-19 worldwide. The currently employed six-month regimen, consisting of isoniazid, rifampin, pyrazinamide and ethambutol, has high efficacy against drug-sensitive TB, but its length and complexity contributes to treatment interruptions that jeopardize cure and promote drug resistance. Although novel, treatment-shortening antibiotic regimens have shown promising results in international clinical trials, the infrastructure needed to ensure adherence to daily treatment and the associated costs may still pose barriers to their implementation in TB-endemic countries. Recent work has focused on adjunctive, host-directed strategies to simplify and shorten the course of TB therapy.

The need for prolonged TB treatment is believed to reflect the unique ability of a subpopulation of Mycobacterium tuberculosis (Mtb) bacilli within the infected host to remain in a nonreplicating, persistent state characterized by tolerance to first-line anti-TB drugs, like isoniazid (INH), which more effectively targets actively dividing bacilli. One of the key bacterial pathways implicated in antibiotic tolerance is the stringent response, which is regulated by the (p)ppGpp synthase/hydrolase, Rel_{Mtb}. Rel_{Mtb} deficiency results in defective Mtb survival under nutrient starvation, in mouse lungs and mouse hypoxic granulomas, reduced virulence in guinea pigs and C3HeB/FeJ mice, and increased susceptibility of Mtb to isoniazid in mouse lungs, rendering Rel_{Mtb} an attractive target for novel antitubercular therapies, including for drug-resistant TB.

As such, there exists an unmet need for continued rel_{Mtb} vaccine development to therapeutically and/or prophylactically address Mtb infections.

### SUMMARY OF THE INVENTION

This disclosure describes compositions, methods, kits, and related aspects for providing prophylaxis and/or reducing treatment times for Mycobacterium tuberculosis (Mtb) infections. In some aspects, for example, the present disclosure provides nucleic acid vaccine constructs involving fusion of the gene encoding rel_{Mtb} with the gene encoding the immature dendritic cell-targeting chemokine MIP-3α/CCL20 (MIP-3α/rel_{Mtb} or “fusion vaccine”). In some embodiments, the present disclosure provides nucleic acid vaccine constructs involving fusion of the gene encoding rel_{Mtb} with a gene encoding another chemokine that binds to a chemokine receptor 6 (CCR6) or with a gene encoding an antibody, or antigen binding portion thereof, that binds to a CCR6. In some embodiments, intranasal immunization with these nucleic acid vaccines expressing MIP-3α/rel_{Mtb}, generate robust, immune responses and enhance mycobactericidal activity when combined with antibiotic agents, such as isoniazid (INH). These and other aspects will be apparent upon complete review of the present disclosure, including the accompanying figures.

In one aspect, the present disclosure provides a nucleic acid vaccine (e.g., a DNA vaccine, an mRNA vaccine, etc.) composition comprising a synthetic polynucleotide encoding a Mycobacterium tuberculosis (Mtb) RelA-SpoT homolog (RSH) protein, Rel_{Mtb}, or a functional portion, fragment, or variant thereof, conjugated to a macrophage inflammatory protein-3 alpha (MIP-3α) or other chemokine that binds to a chemokine receptor 6 (CCR6), or a functional portion, fragment, or variant thereof, or to an antibody, or antigen binding portion thereof, that binds to a CCR6. In some embodiments, the synthetic polynucleotide comprises the nucleotide sequence of SEQ ID. NOS: 1 and 3. In some embodiments, the synthetic polynucleotide further comprises the nucleotide sequence of SEQ ID. NO: 2. In some embodiments, the MIP-3α is murine or human. In some embodiments, the synthetic polynucleotide is codon-optimized for expression in a mammalian cell. In some embodiments, the mammalian cell is a human cell.

In some embodiments, a recombinant nucleic acid vector encoding the nucleic acid vaccine compositions is provided. In some embodiments, the vector is a pSectag2B plasmid or a pVax1 plasmid (e.g., comprising an IgE signal peptide). Other vectors are optionally utilized. In some embodiments, a pharmaceutical composition comprising a recombinant nucleic acid vector as disclosed herein and a pharmaceutically acceptable carrier is provided. In some embodiments, the pharmaceutically acceptable carrier comprises a lipid nanoparticle (LNP), a polymeric nanoparticle, a lipidoid, a liposome, a lipoplex, a peptide carrier, a nanoparticle mimic, or a conjugate thereof. In some embodiments, the pharmaceutical compositions further include at least one additional biologically active agent (e.g., an antibiotic agent or the like).

In another aspect, the present disclosure provides a method of providing prophylaxis to, and/or treating an Mtb infection in, a subject in need thereof comprising administering to the subject an effective amount of a composition disclosed herein. In some embodiments, the composition is administered to the subject prior to, concurrent with, and/or after administering at least one antibiotic agent to the subject. In some embodiments, the composition is administered as one or more boost doses after an initial administration of the composition to the subject. In some embodiments, the composition is administered intramuscularly and/or intranasally to the subject.

In another aspect, the present disclosure provides a vaccine composition, comprising a polypeptide that comprises a Mycobacterium tuberculosis (Mtb) RelA-SpoT homolog (RSH) protein, Rel_{Mtb}, or a functional portion, fragment, or variant thereof, conjugated to a macrophage inflammatory protein-3 alpha (MIP-3α) or other chemokine that binds to a chemokine receptor 6 (CCR6), or a functional portion, fragment, or variant thereof, or to an antibody, or antigen binding portion thereof, that binds to a CCR6. In some embodiments, the polypeptide comprises the amino acid sequence of SEQ ID. NOS: 5 and 7. In some embodiments, the polypeptide further comprises the amino acid sequence of SEQ ID. NO: 6. In some embodiments, the present disclosure provides a pharmaceutical composition comprising a vaccine composition disclosed herein and a pharmaceutically acceptable carrier. In some of these embodiments, the pharmaceutical composition further comprises at least one additional biologically active agent.

In some of these embodiments, the present disclosure provides a method of providing prophylaxis to, and/or treating an Mtb infection in a subject in need thereof comprising administering to the subject an effective amount of the vaccine composition. In some embodiments, the composition is administered to the subject prior to, concurrent with, and/or after administering at least one antibiotic agent to the subject. In some of these embodiments, the composition is administered as one or more boost doses after an initial administration of the composition to the subject. In some of these embodiments, the composition is administered intramuscularly and/or intranasally to the subject.

### BRIEF DESCRIPTION OF THE DRAWINGS

FIGS. 1A-1C. MIP-3α fusion and IN delivery of vaccine expressing rel_{Mtb} increase the mycobactericidal activity of INH in a murine model of chronic TB. (A) Diagrammatic representation of the MIP-3α/rel_{Mtb} and rel_{Mtb} DNA constructs used for immunization. (B) Timeline of the Mtb challenge study, (C) Scatterplot of lung mycobacterial burden at 10 weeks after the primary vaccination per vaccination group: IN delivery of a DNA vaccine expressing rel_{Mtb} or IM delivery of a DNA vaccine expressing MIP-3α/rel_{Mtb} enhances the mycobactericidal activity of INH in vivo compared to IM delivery of rel_{Mtb} vaccine. The greatest therapeutic effect is demonstrated after IN MIP-3α/rel_{Mtb} vaccination, which is more efficacious compared to any other group. Mtb: Mycobacterium tuberculosis, IM: Intramuscular, IN: Intranasal, SD: Standard Dose, HD: High Dose, CFU: colony-forming units, INH: Isoniazid.

FIGS. 2A-2J. T-cell responses in murine tissues 6 weeks after Mtb challenge: IN vaccination with rel_{Mtb} or IM vaccination with MIP-3α/rel_{Mtb} elicits higher Th17 or Th1 response compared to IM vaccination with rel_{Mtb}, while IN vaccination with MIP-3α/rel_{Mtb} offers the most robust systemic and local Th1 and Th17 responses of all experimental groups. Rel_{Mtb}-specific IFN-γ-producing CD4+ T cells (A) and CD8+ T cells (B) in spleens; Rel_{Mtb}-specific IFN-γ-producing CD4+ T cells (C) and CD8+ T cells (D) in lungs; Rel_{Mtb}-specific, TNF-α-producing CD4+ T cells (E) and CD8+ T cells (F) in spleens; Rely-specific TNF-α-producing CD4+ T cells (G) and CD8+ T cells (H) in lungs; rel_{Mtb}-specific, IL-17α-producing CD4+ T cells in lungs (I) and spleens (J) (flow cytometry intracellular staining). IM: Intramuscular (standard dose is shown), IN: Intranasal (high dose is shown). Y-axis scales are different among cytokines and between tissues in order to better demonstrate differences between groups where cytokine expression levels were lower.

FIGS. 3A-3D. T-cell responses in non-infected murine tissues 6 weeks after prime vaccination: IN vaccination with rel_{Mtb} or MIP-3α/rel_{Mtb} and IM vaccination with MIP-3α/rel_{Mtb} elicited stronger IL-17a CD4+ T-cell responses in cells extracted from draining LNs, PBMCs and higher TNF-α CD4+ T cells in PBMCs compared to IM vaccination with rel_{Mtb}. (A) Timeline of the immunogenicity study. Rel_{Mtb}-specific, IL-17α-producing CD4+ T cells in draining LNs (B) and PBMCs (C) and Rel_{Mtb}-specific, TNF-α-producing CD4+ T cells (D) in PBMCs. (flow cytometry-intracellular staining). IM: Intramuscular, IN: Intranasal, LNs: Lymph nodes, PBMCs: Peripheral Blood Mononuclear Cells. Y-axis scales are different among cytokines and between tissues in order to better demonstrate differences between groups where cytokine expression levels were lower.

FIGS. 4A-4I. IN vaccination with MIP-3α/rel_{Mtb} increases the simultaneous production of multiple cytokines associated with Mtb control, systemically and at the site of infection. IN MIP-3α/rel_{Mtb} vaccination group was found to have the highest aggregate production of the IL17-α, TNF-α, IFN-γ, and IL-2-producing CD4+ and CD8+ T cells in the spleens and lungs of Mtb-infected animals compared to any other group (A, B, C), but also had the highest normalized production of each cytokine individually across experimental groups (D, E, F, G, H, I).

FIGS. 5A-5C. (A) Normalized mean mouse lung weights at 10 weeks (B) Lung mycobacterial burden at: implantation (−4 weeks); initiation of treatment (0 weeks); and at 6 weeks and 10 weeks after the initiation of treatment. (C) Gross pathology of representative lungs per experimental group; black line represents 1 cm, TB: tuberculosis, IM: Intramuscular, IN: Intranasal, SD: Standard Dose, HD: High Dose, CFU: colony-forming units. ***=significant difference from control and INH (at least P<0.001), ** significant difference from control and INH (at least P<0.01), * significant difference from control and INH (at least P<0.05), ####significant difference from control only (P<0.0001).

FIGS. 6A-6L. T-cell responses in non-infected murine tissues after vaccination (immunogenicity, non-challenged, animal study). (A) rel_{Mtb}-specific IFN-γ response in spleens between IM vaccination with MIP-3α/rel_{Mtb} vs rel_{Mtb}, as assessed by FluoroSpot. (B) Representative pictures of FluoroSpot are shown per group. Each triplicate represents three different mice. (C) and (D) rel_{Mtb}-specific TNF-α producing CD4+ and CD8+ T cells in cells derived from spleens among the different vaccination groups as assessed by flow cytometry. (E) and (F) Rel_{Mtb}-specific IL-2 producing CD4+ and CD8+ T cells among in cells derived from spleens among the different vaccination groups as assessed by flow cytometry. (G) rel_{Mtb}-specific IL-17a response in spleens among different vaccination groups as assessed by FluoroSpot. (H) Representative pictures of FluoroSpot are shown per group. Each triplicate represents three different mice. (I) Rel_{Mtb}-specific IL-17a producing CD4+ T cells in cells derived from spleens among the different vaccination groups as assessed by flow cytometry (J) rel_{Mtb}-specific IL-17a producing CD4+ T cells in cells derived from lungs among the different vaccination groups as assessed by flow cytometry (K) and (L) rel_{Mtb}-specific TNF-α producing CD4+ and CD8+ T cells in cells derived from spleens among the different vaccination groups as assessed by flow cytometry. (E) and (F) rel_{Mtb}-specific IFN-γ producing CD4+ and CD8+ T cells in cells derived from lungs among the different vaccination groups as assessed by flow cytometry. SFU: Spot Forming Unit, IM: Intramuscular, IN: Intranasal. Y-axis scales are different among cytokines and between tissues in order to better demonstrate differences between groups where cytokine expression levels were lower.

FIGS. 7A and 7B. Evaluation of T cell lung homing in murine lungs (6 weeks post prime vaccination). (A) CD3+CD4+CXCR3+KLRG1−. (B) CD3+CD8+CXCR3+KLRG1−.

FIG. 8. Evaluation of DC subgroups in murine lungs (6 weeks post prime vaccination; CD3−CD11c+CD103−CD11b+).

FIG. 9. Evaluation of DC subgroups in murine lungs (10 weeks post prime vaccination; CD3−NK1.1−CD19−CD11c+CD103−CD11b+).

FIGS. 10A-10C. Evaluation of DC activation in murine lungs (6 weeks post prime vaccination). (A) CD3−CD11c+CCR6+. (B) CD3−CD11c+CCR7+. (C) CD3−CD11c+CD103−CD11b+MHC II+.

FIGS. 11A and 11B. Evaluation of DC activation in murine lungs (10 weeks post prime vaccination). (A) CD3−NK1.1−CD19−CD11c+MHC II+. (B) CD3−NK1.1−CD19−CD11c+CD80+.

FIG. 12. IgG+IgM+IgA total antibody response in BAL (10 weeks post prime vaccination).

FIG. 13. Second Mtb challenge experiment-6 weeks upon treatment initiation.

FIG. 14. Timecourse plot (y-axis=lung bacterial burden (CFU); x-axis=treatment (weeks)).

FIG. 15. Testing IN fusion-low dose 4 weeks upon tx initiation.

FIG. 16. Normalized lung weight plot (6 weeks of treatment females).

FIGS. 17A and 17B. Rel_{Mtb}-specific CD4+ and CD8+ T cells producing-IFN-γ as assessed by intracellular staining. (A) CD3+CD4+IFN-γ % splenocytes. (B) CD3+CD8+IFN-γ % splenocytes.

FIGS. 18A and 18B. MIP-3α/rel_{Mtb} CD4+ and CD8+ T cells producing-IL-2 as assessed by intracellular staining. (A) CD3+CD4+IL-2% splenocytes. (B) CD3+CD8+IL-2% splenocytes.

FIGS. 19A and 19B. MIP-3α/rel_{Mtb} CD4+ and CD8+ T cells producing-TNF-α as assessed by intracellular staining. (A) CD3+CD4+TNF-α % splenocytes. (B) CD3+CD8+TNF-α % splenocytes.

FIG. 20. ELISA Rel_{Mtb}-specific antibody titers-plasma.

### DETAILED DESCRIPTION OF THE INVENTION

Tuberculosis (TB) is one of the leading causes of death from a single infectious agent worldwide. The lengthy treatment regimen reflects the unique ability of a subpopulation of “persister” bacteria to remain in a nonreplicating state in the infected host through various adaptive strategies, including induction of the stringent response. The key stringent response enzyme, Rel_{Mtb}, is essential for long-term Mycobacterium tuberculosis (Mtb) survival under physiologically relevant stresses in vitro and in animal lungs. Recently, the present inventors generated a therapeutic, parenteral, rel_{Mtb} DNA vaccine, which induces Rel_{Mtb}-specific cellular immunity and augments the activity of the first-line drug isoniazid against active TB in mice and guinea pigs. In the present disclosure, the inventors provide a novel vaccination strategy involving the fusion of an antigen of interest with the immature dendritic cell (iDC)-targeting chemokine macrophage inflammatory protein-3 alpha (MIP-3α or CCL20), which significantly enhances antigen-specific T-cell responses. As described herein, this iDC-targeting strategy improves the immunogenicity of the therapeutic rel_{Mtb} DNA vaccine.

In some aspects of the present disclosure, rel_{Mtb} DNA and chemokine MIP-3α are cloned into eukaryotic expression vectors, such as plasmid pSectag2b or a pVAX1 plasmid. As described in an Example provided herein, an immunogenicity study was conducted using C57BL/6J mice, comparing the T-cell responses between the rel_{Mtb} vs. MIP-3α/rel_{Mtb} DNA intramuscular vaccination groups. The results of this study show, for example, that intramuscular administration of the MIP-3α/rel_{Mtb} vaccines of the present disclosure induced increased production of various Mtb-protective cytokines (IL-17α, IL-2, TNF-α, IFN-γ) in various mouse tissues, including spleen, draining lymph nodes and peripheral blood mononuclear cells, relative to the rel_{Mtb} vaccine. That is, intramuscular therapeutic immunization with the DNA vaccine expressing MIP-3α/rel_{Mtb} induces promising Mtb-protective immune signatures in vivo compared to rel_{Mtb} vaccine.

Other routes of administering the DNA vaccine of the present disclosure have also been evaluated. As further described herein, for example, to augment mucosal immune responses to this respiratory pathogen, the intranasal vaccination route was also studied. Intranasal immunization with the DNA vaccines of the present disclosure, which express MIP-3α/rel_{Mtb}, generated robust, polyfunctional Th1/Th17 responses and offered the greatest mycobactericidal activity when combined with INH. Accordingly, in some aspects, this DNA vaccination strategy is a promising adjunctive approach combined with standard therapy to shorten curative TB treatment.

Therefore, in accordance with an embodiment, the present disclosure provides a nucleic acid vaccine composition comprising a synthetic polynucleotide encoding a Mycobacterium tuberculosis (Mtb) RelA-SpoT homolog (RSH) protein, Rel_{Mtb}, or a functional portion, fragment, or variant thereof, conjugated (e.g., directly or via a linker or spacer nucleotide sequence) to a synthetic polynucleotide encoding a macrophage inflammatory protein-3 alpha (MIP-3α) or other chemokine that binds (e.g., specifically binds) to a chemokine receptor 6 (CCR6), or a functional portion, fragment, or variant thereof, or to a synthetic polynucleotide encoding an antibody, or antigen binding portion thereof, that binds (e.g., specifically binds) to a CCR6. As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope. As used herein, the term “antigen binding portion” refers to a portion of an antibody that binds to a chemokine receptor 6 (CCR6), e.g., a molecule in which one or more immunoglobulin chains is not full length, but which binds to CCR6. Examples of binding portions encompassed within the term “antigen-binding portion” of an antibody include (i) a Fab fragment, a monovalent fragment consisting of the VLC, VHC, CL and CHI domains: (ii) a F(ab′)2 fragment, a bivalent fragment comprising two Fab fragments linked by a disulfide bridge at the hinge region; (iii) a Fd fragment consisting of the VHC and CHI domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody binding portions are obtained using conventional techniques known to those with skill in the art, and the portions are screened for utility in the same manner as are intact antibodies.

In some embodiments, the synthetic polynucleotide comprises the nucleotide sequence of SEQ ID. NOS: 1, 2, and 3 (shown below in Table 1 (Mouse Codon Optimized MIP-3α/Rel_{Mtb} DNA Sequences)) or comprises a polynucleotide having at least 80%, 85%, 90%, 95%, 99% sequence identity with SEQ ID NOS: 1, 2, and 3. Table 2 shows a non-codon optimized Rel_{Mtb} DNA sequence. The phrase “functional portion, fragment, or variant thereof” in the context of the proteins described herein, refers to a portion or fragment of the full-length protein or a non-wild-type form of the protein (full-length, portion, or fragment thereof) that retains a desired property or function, such as targeting a cell type (e.g., an iDC), improving immunogenicity, effectuating vaccination, or the like. In some embodiments, vaccine compositions of the present disclosure comprise polypeptides that comprises a Rel_{Mtb}, or a functional portion, or fragment or variant thereof, conjugated to MIP-3α, or a functional portion or fragment or variant thereof (e.g., a MIP-3a rel_{Mtb} fusion protein).

TABLE 1

| | SEQ | |
|---|---|---|
| | ID | |
| DNA SEQUENCE (Top Strand Bases) | NO | Description |
| GCCGCTAGCAACTTCGACTGCTGTCTGGGA | 1 | MIP-3α |
| TACACAGATAGAATCCTGCACCCAAAGTTC | | |
| ATCGTGGGCTTTACCAGACAGCTGGCCAAC | | |
| GAGGGATGCGACATCAACGCTATCATCTTT | | |
| CACACCAAGAAGAAGCTGAGCGTGTGCGCC | | |
| AACCCCAAGCAGACATGGGTGAAGTACATC | | |
| GTGCGGCTGCTGAGCAAGAAGGTGAAGAAC | | |
| ATG | | |
| GGACCAGGACCTGGACCAGGACCAGGACCT | 2 | Linker |
| CAGGCGCCGAAGAGTCTCGAGGCTAGC | | |
| ACCGCCCAGAGGTCTACCACAAACCCTGTG | 3 | Mouse |
| CTGGAGCCACTGGTGGCAGTCCACAGGGAG | | codon |
| ATCTACCCCAAGGCCGATCTGAGCATCCTG | | optimized |
| CAGAGGGCATATGAGGTGGCAGACCAGAGG | | Rel_{Mtb} |
| CACGCCAGCCAGCTGCGCCAGTCCGGCGAT | | |
| CCTTACATCACACACCCACTGGCCGTGGCC | | |
| AATATCCTGGCCGAGCTGGGCCTGGACACC | | |
| ACAACCCTGGTGGCCGCCCTGCTGCACGAC | | |
| ACCGTGGAGGATACAGGCTATACCCTGGAG | | |
| GCCCTGACAGAGGAGTTCGGAGAGGAAGTG | | |
| GGACACCTGGTGGACGGAGTGACCAAGCTG | | |
| GATAGGGTGGTGCTGGGCTCCGCCGCAGAG | | |
| GGAGAGACAATCAGAAAGATGATCACAGCA | | |
| ATGGCCAGGGACCCCAGGGTGCTGGTCATC | | |
| AAGGTGGCCGACCGGCTGCACAACATGAGG | | |
| ACCATGAGATTCCTGCCACCTGAGAAGCAG | | |
| GCAAGGAAGGCCAGGGAGACACTGGAAGTG | | |
| ATCGCACCACTGGCCCACAGGCTGGGAATG | | |
| GCCTCTGTGAAGTGGGAGCTGGAGGACCTG | | |
| AGCTTTGCCATCCTGCACCCTAAGAAGTAC | | |
| GAGGAGATCGTGCGGCTGGTGGCAGGAAGG | | |
| GCACCAAGCAGAGATACCTATCTGGCCAAG | | |
| GTGCGCGCCGAGATCGTGAATACACTGACC | | |
| GCCTCTAAGATCAAGGCCACAGTGGAGGGC | | |
| AGGCCCAAGCACTACTGGAGCATCTATCAG | | |
| AAGATGATCGTGAAGGGCAGAGACTTCGAC | | |
| GATATCCACGATCTGGTGGGCGTGAGAATC | | |
| CTGTGCGACGAGATCCGCGATTGTTACGCA | | |
| GCAGTGGGAGTGGTGCACAGCCTGTGGCAG | | |
| CCAATGGCAGGCCGGTTTAAGGACTATATC | | |
| GCCCAGCCCCGCTACGGCGTGTATCAGTCC | | |
| CTGCACACAACCGTGGTGGGACCAGAGGGC | | |
| AAGCCTCTGGAGGTGCAGATCCGGACCCGC | | |
| GATATGCACAGGACAGCAGAGTACGGAATC | | |
| GCAGCACACTGGAGGTATAAGGAGGCCAAG | | |
| GGCAGAAACGGCGTGCTGCACCCTCACGCA | | |
| GCAGCAGAGATCGACGATATGGCCTGGATG | | |
| AGGCAGCTGCTGGACTGGCAGAGGGAGGCA | | |
| GCCGATCCCGGAGAGTTCCTGGAGTCTCTG | | |
| CGCTACGACCTGGCCGTGCAGGAGATCTTC | | |
| GTGTTTACCCCTAAGGGCGACGTGATCACA | | |
| CTGCCCACCGGCAGCACACCTGTGGATTTT | | |
| GCCTATGCAGTGCACACAGAAGTGGGACAC | | |
| AGGTGCATCGGAGCCCGGGTGAACGGCCGC | | |
| CTGGTGGCCCTGGAGCGCAAGCTGGAGAAT | | |
| GGCGAGGTGGTGGAGGTGTTTACCAGCAAG | | |
| GCACCAAACGCAGGACCCTCCAGAGACTGG | | |
| CAGCAGTTCGTGGTGTCCCCAAGGGCCAAG | | |
| ACCAAGATCAGACAGTGGTTTGCCAAGGAG | | |
| AGGAGAGAGGAGGCCCTGGAGACAGGCAAG | | |
| GATGCCATGGCCCGGGAGGTGCGGAGGGGA | | |
| GGCCTGCCCCTGCAGCGCCTGGTGAATGGA | | |
| GAGTCTATGGCAGCAGTGGCCAGGGAGCTG | | |
| CACTACGCAGACGTGAGCGCCCTGTATACC | | |
| GCAATCGGAGAGGGACACGTGTCCGCCAAG | | |
| CACGTGGTGCAGAGACTGCTGGCCGAGCTG | | |
| GGAGGAATCGATCAGGCCGAGGAGGAGCTG | | |
| GCCGAGAGGTCTACCCCAGCCACAATGCCC | | |
| AGGAGGCCCAGATCTACCGACGATGTGGGC | | |
| GTGAGCGTGCCAGGAGCACCAGGCGTGCTG | | |
| ACCAAGCTGGCCAAGTGCTGTACACCAGTG | | |
| CCCGGCGACGTGATCATGGGATTCGTGACA | | |
| AGGGGCGGAGGCGTGTCCGTGCACAGAACC | | |
| GATTGTACAAACGCAGCCTCTCTGCAGCAG | | |
| CAGGCAGAGAGGATCATCGAGGTGCTGTGG | | |
| GCCCCTTCCCCAAGCTCCGTGTTTCTGGTG | | |
| GCCATCCAGGTGGAGGCCCTGGACAGGCAC | | |
| AGACTGCTGTCTGATGTGACCAGAGCCCTG | | |
| GCCGACGAGAAAGTGAATATCCTGTCTGCC | | |
| AGCGTGACAACCTCCGGCGACAGGGTGGCC | | |
| ATCAGCAGGTTCACCTTCGAGATGGGCGAT | | |
| CCTAAGCACCTGGGCCACCTGCTGAACGCC | | |
| GTGAGGAATGTGGAGGGCGTGTACGACGTG | | |
| TATAGAGTGACCTCCGCCGCC | | |

TABLE 2

| | SEQ | |
|---|---|---|
| | ID | |
| DNA SEQUENCE (Top Strand Bases) | NO | Description |
| GTGGCCGAGGACCAGCTCACGGCGCAAGCGG | 4 | Non- |
| TTGCACCGCCCACGGAGGCTTCTGCGGCTC | | codon |
| TCGAGCCCGCTCTCGAGACGCCCGAGTCGC | | optimized |
| CGGTCGAGACTCTTAAGACCAGCATCAGCG | | Rel_{Mtb} |
| CGTCGCGTCGGGTGCGGGCCCGATTGGCCC | | |
| GGCGGATGACCGCCCAGCGCAGCACCACCA | | |
| ATCCGGTGCTCGAGCCGTTGGTGGCGGTGC | | |
| ACCGGGAGATCTATCCCAAGGCCGACCTGT | | |
| CGATCTTGCAGCGAGCCTACGAGGTCGCTG | | |
| ACCAAAGGCATGCCAGCCAGTTGCGGCAGT | | |
| CCGGTGATCCCTACATCACCCACCCGTTGG | | |
| CCGTTGCCAACATTCTGGCCGAGTTGGGCA | | |
| TGGACACCACCACTTTGGTGGCCGCGCTGC | | |
| TGCACGACACCGTCGAGGACACCGGTTACA | | |
| CCCTGGAGGCGTTGACCGAGGAATTCGGCG | | |
| AAGAGGTGGGCCATCTCGTCGACGGGGTGA | | |
| CCAAGCTGGATCGGGTGGTGTTGGGCAGCG | | |
| CCGCCGAAGGCGAGACTATTCGCAAGATGA | | |
| TCACCGCGATGGCCCGCGATCCGCGGGTGC | | |
| TGGTGATAAAGGTGGCTGACCGGTTACACA | | |
| ACATGCGCACCATGCGCTTCTTGCCGCCGG | | |
| AGAAGCAGGCCCGCAAGGCCCGTGAGACGT | | |
| TGGAAGTCATTGCACCCCTGGCGCATCGGC | | |
| TGGGCATGGCCAGCGTCAAGTGGGAGTTGG | | |
| AGGACCTGTCCTTCGCGATCCTGCATCCCA | | |
| AGAAGTACGAGGAGATCGTCCGGCTGGTCG | | |
| CCGGTCGCGCGCCGTCCCGGGACACCTACC | | |
| TGGCCAAGGTGCGTGCCGAAATCGTCAACA | | |
| CGCTGACCGCGTCGAAGATCAAGGCGACGG | | |
| TGGAGGGCCGCCCCAAGCACTATTGGTCGA | | |
| TCTACCAGAAGATGATCGTTAAGGGCCGCG | | |
| ACTTCGACGACATCCACGACCTGGTCGGTG | | |
| TGCGCATCCTGTGCGACGAAATCCGGGACT | | |
| GCTACGCGGCTGTCGGCGTAGTGCATTCGC | | |
| TATGGCAGCCGATGGCGGGTCGGTTCAAGG | | |
| ACTACATCGCCCAGCCCAGATACGGTGTGT | | |
| ACCAGTCACTGCACACCACTGTGGTCGGGC | | |
| CTGAGGGCAAGCCGCTGGAAGTGCAGATCC | | |
| GTACCCGCGACATGCACCGCACCGCCGAAT | | |
| ACGGCATCGCCGCGCATTGGCGCTACAAAG | | |
| AAGCCAAGGGCCGCAACGGTGTTCTTCATC | | |
| CGCATGCCGCCGCGGAGATCGACGACATGG | | |
| CCTGGATGCGTCAGCTGCTCGACTGGCAAC | | |
| GTGAGGCGGCCGACCCCGGTGAGTTCTTGG | | |
| AATCATTGCGCTACGACCTTGCGGTGCAAG | | |
| AGATTTTCGTGTTTACCCCCAAGGGCGACG | | |
| TGATCACGCTGCCAACCGGTTCGACGCCGG | | |
| TGGACTTCGCTTACGCGGTGCACACAGAGG | | |
| TGGGCCACCGCTGCATCGGCGCCCGAGTGA | | |
| ACGGCCGGTTGGTAGCGCTGGAACGCAAGC | | |
| TGGAAAACGGAGAAGTTGTCGAGGTTTTCA | | |
| CGTCCAAGGCGCCGAACGCCGGGCCGTCGC | | |
| GGGACTGGCAGCAGTTCGTGGTGTCGCCGC | | |
| GCGCAAAGACGAAGATCCGCCAGTGGTTCG | | |
| CCAAGGAGCGGCGTGAGGAGGCGTTGGAGA | | |
| CCGGTAAGGATGCGATGGCCCGCGAGGTGC | | |
| GCCGCGGTGGACTTCCGTTGCAGCGCTTGG | | |
| TCAATGGTGAGTCCATGGCGGCGGTGGCCC | | |
| GCGAGCTGCACTACGCGGACGTGTCAGCAC | | |
| TCTATACCGCCATCGGTGAGGGGCACGTGT | | |
| CGGCGAAACACGTCGTGCAGCGGTTGTTGG | | |
| CCGAGCTCGGCGGTATCGACCAGGCGGAAG | | |
| AGGAACTCGCCGAGCGGTCCACGCCGGCGA | | |
| CCATGCCGCGGCGCCCACGCAGCACCGACG | | |
| ATGTCGGGGTCTCCGTCCCCGGCGCCCCGG | | |
| GCGTGCTGACCAAGCTGGCCAAGTGCTGCA | | |
| CGCCGGTTCCGGGCGATGTGATTATGGGGT | | |
| TCGTCACCCGTGGCGGCGGGGTCAGTGTGC | | |
| ACCGCACCGACTGCACCAACGCCGCATCGC | | |
| TGCAGCAGCAGGCCGAGCGCATCATCGAGG | | |
| TGCTATGGGCGCCGTCGCCGTCGTCGGTGT | | |
| TTCTGGTGGCAATCCAGGTCGAGGCACTCG | | |
| ACCGGCACCGGCTGCTGTCGGATGTGACGC | | |
| GCGCACTGGCCGACGAGAAGGTCAATATCC | | |
| TGTCCGCGTCGGTCACCACTTCGGGGGACC | | |
| GGGTGGCGATCAGTCGATTCACCTTCGAGA | | |
| TGGGTGACCCCAAGCACCTCGGGCACCTGC | | |
| TCAACGCCGTCCGCAACGTCGAAGGTGTCT | | |
| ACGACGTCTACCGGGTGACCTCGGCCGCG | | |

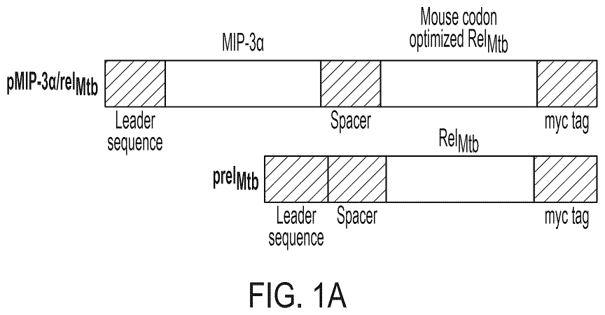

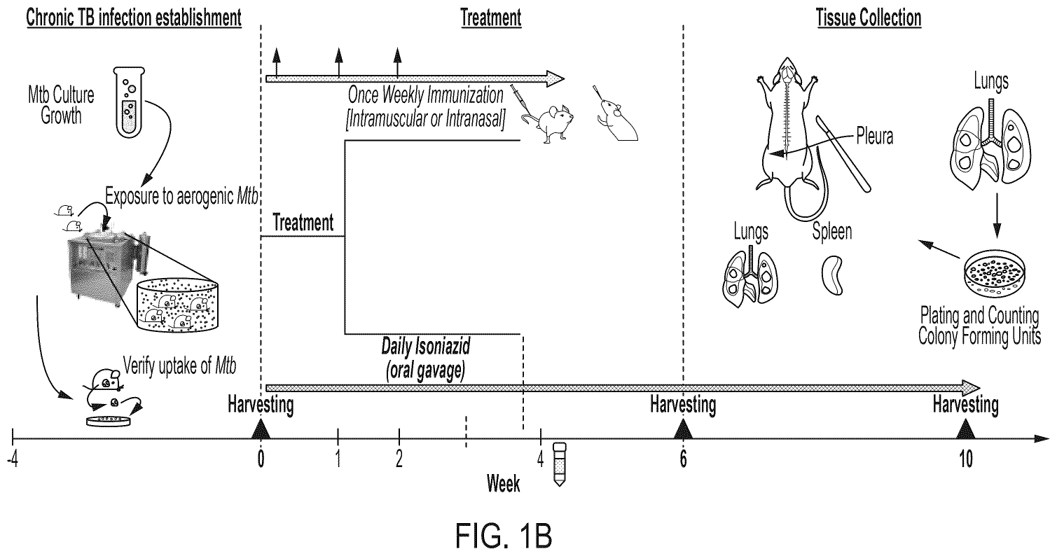

In some embodiments, synthetic polynucleotide encoding Rel_{Mtb}, or a functional portion, fragment, or variant thereof, conjugated to MIP-3α, or a functional portion, fragment, or variant thereof include other nucleic acid elements, such as leader sequences, spacers or linkers, tags, and/or the like. An example of such a synthetic polynucleotide configuration is shown in FIG. 1A. In some embodiments, the linker comprises a polynucleotide which encodes one or more amino acids. The linker can be a polynucleotide encoding 1 to 50 amino acids, including, for example, 2, 3, 4, 5, 10, 15, 20, 30, 40, up to 50 amino acids. In an embodiment, the linker is a dipeptide. In another embodiment, the linker is the dipeptide Glu-Phe. Typically, the synthetic polynucleotides of the present disclosure are included as expression cassettes in a recombinant nucleic acid vector, such as a plasmid (e.g., a pSectag2B plasmid, a pVAX1 plasmid, etc.) or the like. Exemplary expression vectors are described further herein.

As used herein, the term “Mtb RelA-SpoT homolog (RSH) protein” or “Rel_{Mtb}” refers to a bifunctional Rel/SpoT homolog (RSH) protein encoded by M. tuberculosis. Unlike members of the γ- and β-proteobacteria lineages, which encode two functionally divergent RSH homologs (RelA and SpoT), Mtb encodes a single bifunctional RSH enzyme, Rel_{Mtb}, which is conserved in all Mycobacterium species. Rel_{Mtb} contains two catalytic domains, a (p)ppGpp hydrolysis domain (1 to 181 amino acids) and a (p)ppGpp synthetase domain (87 to 394 amino acids), and a regulatory C-terminal domain (395 to 738 amino acids). The synthesis of ppGpp and pppGpp is catalyzed by the (p)ppGpp synthetase domain through transfer of the 5′-β, γ-pyrophosphate from adenosine 5′-triphosphate (ATP) to the 3′-OH of guanosine diphosphate (GDP) or guanosine 5′-triphosphate (GTP), respectively. Crystallography studies showed that the Mtb (p)ppGpp synthetase domain comprises five β sheets surrounded by five α helices, and mutational analysis revealed that amino acids D265 and E325 are required for (p)ppGpp synthesis in vitro. The (p)ppGpp hydrolysis domain comprises 11 α helices, including a (p)ppGpp-binding pocket between the second and the third a helices, and amino acids H80 and D81 are critical for hydrolase activity but dispensable for (p)ppGpp synthesis. The function of each Rel_{Mtb} catalytic domain is dependent on the concentration of cation cofactors, including Mg²⁺ and Mn². Although rel_{Mtb} is constitutively expressed at basal levels, (p)ppGpp synthetase activity is repressed by the C-terminal domain in the absence of stresses, and (p)ppGpp accumulates in Mtb during NS and in response to hypoxia and oxidative stress. The preponderance of evidence suggests that the classic model of (p)ppGpp affecting RNA polymerase promoter open complexes to alter gene expression during the stringent response may be conserved in Mtb, but the underlying molecular mechanisms may differ from those of model organisms. Additional details regarding Rel_{Mtb} are provided in, for example, Avarbock et al., “Functional regulation of the opposing (p)ppGpp synthetase/hydrolase activities of RelMtb from Mycobacterium tuberculosis,” Biochemistry, 2005, 44:9913-9923, which in incorporated by reference.

As used herein, the term “macrophage inflammatory protein 3a” or “MIP-3α” (also known as chemokine (C-C motif) ligand 20 (CCL20) or liver activation regulated chemokine (LARC)) refers to a small cytokine belonging to the CC chemokine family. The protein attracts memory T cells and natural killer cells to sites of inflammation, as well as immature dendritic cells. MIP-3α is implicated in the formation and function of mucosal lymphoid tissues via chemoattraction of lymphocytes and dendritic cells towards the epithelial cells surrounding these tissues. In addition, MIP-3α elicits its effects on its target cells by binding and activating the chemokine receptor CCR6. In some embodiments, the MIP-3α can be murine, porcine, ovine, bovine, human, or combinations thereof. It will be understood by those of ordinary skill in the art that many different isoforms of these exist and are optionally used in the compositions disclosed herein. Examples of some of these include Homo sapiens (human) Gene ID: 6364 and Mus musculus (house mouse) Gene ID: 20297.

The synthetic polynucleotides of the nucleic acid vaccine (e.g., DNA vaccines, mRNA vaccines, etc.) compositions of the present disclosure can be comprised within an expression cassette. The term “expression cassette” or “expression vector” as used herein refers to a nucleotide sequence, which is capable of affecting expression of a protein coding sequence in a host compatible with such sequences. Expression cassettes typically include at least a promoter operably linked with the polypeptide coding sequence; and, optionally, with other sequences, e.g., transcription termination signals. Additional factors necessary or helpful in effecting expression may also be included, e.g., enhancers. “Operably linked”, refers to linkage of a promoter upstream from a DNA sequence such that the promoter mediates transcription of the DNA sequence. Thus, expression cassettes include plasmids, recombinant viruses, any form of a recombinant “naked DNA” vector, and the like. In some embodiments, expression cassettes include elements that have been codon optimized for expression in the intended host.

The term “immunogen” or “immunogenic composition” is synonymous with “antigen or antigenic” and refers to a compound or composition comprising a peptide, polypeptide or protein which is “immunogenic,” i.e., capable of eliciting, augmenting or boosting a cellular and/or humoral immune response, either alone or in combination or linked or fused to another substance. An immunogenic composition can be a peptide of at least about 5 amino acids, a peptide of 10 amino acids in length, a fragment 15 amino acids in length, a fragment 20 amino acids in length or greater; smaller immunogens may require presence of a “carrier” polypeptide e.g., as a fusion protein, aggregate, conjugate or mixture, preferably linked (chemically or otherwise) to the immunogen. The immunogen can be recombinantly expressed from a vaccine vector, which can be naked DNA comprising the immunogen's coding sequence operably linked to a promoter, e.g., an expression cassette. The immunogen includes one or more antigenic determinants or epitopes, which may vary in size from about 3 to about 15 amino acids. In some embodiments, the immunogen or antigen is a polypeptide comprising a MIP-3α/rel_{Mtb} fusion protein as described herein.

In accordance with some embodiments, the present disclosure provides a recombinant vector encoding the nucleic acid vaccine compositions described herein. By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions, such as when a given polynucleotide encodes a functional portion, fragment, or variant of Rel_{Mtb} and/or MIP-3α.

Preferably, the nucleic acids of the present disclosure are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine-substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

In some embodiments, the substituted nucleic acid sequence may be optimized. Without being bound to a particular theory, it is believed that optimization of the nucleic acid sequence increases the translation efficiency of the mRNA transcripts. Optimization of the nucleic acid sequence may involve substituting a native codon for another codon that encodes the same amino acid, but can be translated by tRNA that is more readily available within a cell, thus increasing translation efficiency. Optimization of the nucleic acid sequence may also reduce secondary mRNA structures that would interfere with translation, thus increasing translation efficiency. In some embodiments, codon optimization is performed using Genscript.

In some embodiments, the present disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleic acids of the present disclosure can be incorporated into a recombinant expression vector. In this regard, the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally occurring as a whole. However, parts of the vectors can be naturally occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally occurring, non-naturally occurring internucleotide linkages, or both types of linkages. Preferably, the non-naturally occurring or altered nucleotides or internucleotide linkages does not hinder the transcription or replication of the vector.

In some embodiments, the nucleic acid vaccine composition or expression cassette will be inserted into a DNA vector or plasmid. The recombinant expression vector of the present disclosure can be any suitable recombinant expression vector, and can be used to transform or transfect any suitable host. Suitable vectors include those designed for propagation and expansion or for expression or both, such as plasmids and viruses. The vector can be selected from the group consisting of the pSectag2B or pVAX1 series (ThermoFisher Scientific, Carlsbad, CA), the pUC series (Fermentas Life Sciences), the pBluescript series (Stratagene, LaJolla, CA), the pET series (Novagen, Madison, WI), the pGEX series (Pharmacia Biotech, Uppsala, Sweden), pcDNA3 family of plasmids, the pNGVL4a plasmid, and the pEX series (Clontech, Palo Alto, CA). Bacteriophage vectors, such as ΔGT10, ΔGT11, ΔZapII (Stratagene), ΔEMBL4, and ΔNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). The recombinant expression vectors of the present disclosure can be prepared using standard recombinant DNA techniques well known to persons having ordinary skill in the art. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

Desirably, the recombinant expression vector comprises regulatory sequences, such as transcription and translation initiation and termination codons, which are specific to the type of host (e.g., mammalian, bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the expression vectors disclosed herein may include, for instance, neomycin/G418 resistance genes, hygromycin resistance genes, histidinol resistance genes, tetracycline resistance genes, and ampicillin resistance genes, among others.

Recombinant expression vectors can comprise a native or nonnative promoter operably linked to the nucleotide sequence encoding the fusion proteins, polypeptide, or protein (including functional portions and functional variants thereof), or to the nucleotide sequence which is complementary to or which hybridizes to the nucleotide sequence encoding the fusion proteins, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

In accordance with an embodiment, the present invention provides a composition comprising a polypeptide encoding a MIP-3α/rel_{Mtb} fusion protein, or a functional portion, fragment, variant thereof. It will be understood by persons of ordinary skill in the art that the polypeptide encoding a MIP-3α/rel_{Mtb} fusion protein, or a functional portion, fragment, variant thereof is a fusion polypeptide which acts as an immunogen to the immune system and is expressed in the cells of the subject that have taken up the nucleic acid vaccine of the present invention. In some embodiments, the synthetic polypeptide comprises the amino acid sequence of SEQ ID. NOS: 5, 6, and 7 (shown below in Table 3 (Mouse Codon Optimized MIP-3α/Rel_{Mtb} Amino Acid Sequences)) or comprises a polypeptide having at least 80%, 85%, 90%, 95%, 99% sequence identity with SEQ ID NOS: 5, 6, and 7. Table 4 shows a non-codon optimized Rel_{Mtb} amino acid sequence.

TABLE 3

| | SEQ | |
|---|---|---|
| | ID | |
| AMINO ACID SEQUENCE | NO | Description |
| Ala Ala Ser Asn Phe Asp Cys Cys | 5 | MIP-3α |
| Leu Gly Tyr Thr Asp Arg Ile Leu | | |
| His Pro Lys Phe Ile Val Gly Phe | | |
| Thr Arg Gln Leu Ala Asn Glu Gly | | |
| Cys Asp Ile Asn Ala Ile Ile Phe | | |
| His Thr Lys Lys Lys Leu Ser Val | | |
| Cys Ala Asn Pro Lys Gln Thr Trp | | |
| Val Lys Tyr Ile Val Arg Leu Leu | | |
| Ser Lys Lys Val Lys Asn Met | | |
| Gly Pro Gly Pro Gly Pro Gly Pro | 6 | Linker |
| Gly Pro Gln Ala Pro Lys Ser Leu | | |
| Glu Ala Ser | | |
| Thr Ala Gln Arg Ser Thr Thr Asn | 7 | Mouse |
| Pro Val Leu Glu Pro Leu Val Ala | | codon |
| Val His Arg Glu Ile Tyr Pro Lys | | optimized |
| Ala Asp Leu Ser Ile Leu Gln Arg | | Rel_{Mtb} |
| Ala Tyr Glu Val Ala Asp Gln Arg | | |
| His Ala Ser Gln Leu Arg Gln Ser | | |
| Gly Asp Pro Tyr Ile Thr His Pro | | |
| Leu Ala Val Ala Asn Ile Leu Ala | | |
| Glu Leu Gly Leu Asp Thr Thr Thr | | |
| Leu Val Ala Ala Leu Leu His Asp | | |
| Thr Val Glu Asp Thr Gly Tyr Thr | | |
| Leu Glu Ala Leu Thr Glu Glu Phe | | |
| Gly Glu Glu Val Gly His Leu Val | | |
| Asp Gly Val Thr Lys Leu Asp Arg | | |
| Val Val Leu Gly Ser Ala Ala Glu | | |
| Gly Glu Thr Ile Arg Lys Met Ile | | |
| Thr Ala Met Ala Arg Asp Pro Arg | | |
| Val Leu Val Ile Lys Val Ala Asp | | |
| Arg Leu His Asn Met Arg Thr Met | | |
| Arg Phe Leu Pro Pro Glu Lys Gln | | |
| Ala Arg Lys Ala Arg Glu Thr Leu | | |
| Glu Val Ile Ala Pro Leu Ala His | | |
| Arg Leu Gly Met Ala Ser Val Lys | | |
| Trp Glu Leu Glu Asp Leu Ser Phe | | |
| Ala Ile Leu His Pro Lys Lys Tyr | | |
| Glu Glu Ile Val Arg Leu Val Ala | | |
| Gly Arg Ala Pro Ser Arg Asp Thr | | |
| Tyr Leu Ala Lys Val Arg Ala Glu | | |
| Ile Val Asn Thr Leu Thr Ala Ser | | |
| Lys Ile Lys Ala Thr Val Glu Gly | | |
| Arg Pro Lys His Tyr Trp Ser Ile | | |
| Tyr Gln Lys Met Ile Val Lys Gly | | |
| Arg Asp Phe Asp Asp Ile His Asp | | |
| Leu Val Gly Val Arg Ile Leu Cys | | |
| Asp Glu Ile Arg Asp Cys Tyr Ala | | |
| Ala Val Gly Val Val His Ser Leu | | |
| Trp Gln Pro Met Ala Gly Arg Phe | | |
| Lys Asp Tyr Ile Ala Gln Pro Arg | | |
| Tyr Gly Val Tyr Gln Ser Leu His | | |
| Thr Thr Val Val Gly Pro Glu Gly | | |
| Lys Pro Leu Glu Val Gln Ile Arg | | |
| Thr Arg Asp Met His Arg Thr Ala | | |
| Glu Tyr Gly Ile Ala Ala His Trp | | |
| Arg Tyr Lys Glu Ala Lys Gly Arg | | |
| Asn Gly Val Leu His Pro His Ala | | |
| Ala Ala Glu Ile Asp Asp Met Ala | | |
| Trp Met Arg Gln Leu Leu Asp Trp | | |
| Gln Arg Glu Ala Ala Asp Pro Gly | | |
| Glu Phe Leu Glu Ser Leu Arg Tyr | | |
| Asp Leu Ala Val Gln Glu Ile Phe | | |
| Val Phe Thr Pro Lys Gly Asp Val | | |
| Ile Thr Leu Pro Thr Gly Ser Thr | | |
| Pro Val Asp Phe Ala Tyr Ala Val | | |
| His Thr Glu Val Gly His Arg Cys | | |
| Ile Gly Ala Arg Val Asn Gly Arg | | |
| Leu Val Ala Leu Glu Arg Lys Leu | | |
| Glu Asn Gly Glu Val Val Glu Val | | |
| Phe Thr Ser Lys Ala Pro Asn Ala | | |
| Gly Pro Ser Arg Asp Trp Gln Gln | | |
| Phe Val Val Ser Pro Arg Ala Lys | | |
| Thr Lys Ile Arg Gln Trp Phe Ala | | |
| Lys Glu Arg Arg Glu Glu Ala Leu | | |
| Glu Thr Gly Lys Asp Ala Met Ala | | |
| Arg Glu Val Arg Arg Gly Gly Leu | | |
| Pro Leu Gln Arg Leu Val Asn Gly | | |
| Glu Ser Met Ala Ala Val Ala Arg | | |
| Glu Leu His Tyr Ala Asp Val Ser | | |
| Ala Leu Tyr Thr Ala Ile Gly Glu | | |
| Gly His Val Ser Ala Lys His Val | | |
| Val Gln Arg Leu Leu Ala Glu Leu | | |
| Gly Gly Ile Asp Gln Ala Glu Glu | | |
| Glu Leu Ala Glu Arg Ser Thr Pro | | |
| Ala Thr Met Pro Arg Arg Pro Arg | | |
| Ser Thr Asp Asp Val Gly Val Ser | | |
| Val Pro Gly Ala Pro Gly Val Leu | | |
| Thr Lys Leu Ala Lys Cys Cys Thr | | |
| Pro Val Pro Gly Asp Val Ile Met | | |
| Gly Phe Val Thr Arg Gly Gly Gly | | |
| Val Ser Val His Arg Thr Asp Cys | | |
| Thr Asn Ala Ala Ser Leu Gln Gln | | |
| Gln Ala Glu Arg Ile Ile Glu Val | | |
| Leu Trp Ala Pro Ser Pro Ser Ser | | |
| Val Phe Leu Val Ala Ile Gln Val | | |
| Glu Ala Leu Asp Arg His Arg Leu | | |
| Leu Ser Asp Val Thr Arg Ala Leu | | |
| Ala Asp Glu Lys Val Asn Ile Leu | | |
| Ser Ala Ser Val Thr Thr Ser Gly | | |
| Asp Arg Val Ala Ile Ser Arg Phe | | |
| Thr Phe Glu Met Gly Asp Pro Lys | | |
| His Leu Gly His Leu Leu Asn Ala | | |
| Val Arg Asn Val Glu Gly Val Tyr | | |
| Asp Val Tyr Arg Val Thr Ser Ala | | |
| Ala | | |

TABLE 4

| | SEQ | |
|---|---|---|
| | ID | |
| AMINO ACID SEQUENCE | NO | Description |
| Val Ala Glu Asp Gln Leu Thr Ala | 8 | Non- |
| Gln Ala Val Ala Pro Pro Thr Glu | | codon |
| Ala Ser Ala Ala Leu Glu Pro Ala | | optimized |
| Leu Glu Thr Pro Glu Ser Pro Val | | Rel_{Mtb} |
| Glu Thr Leu Lys Thr Ser Ile Ser | | |
| Ala Ser Arg Arg Val Arg Ala Arg | | |
| Leu Ala Arg Arg Met Thr Ala Gln | | |
| Arg Ser Thr Thr Asn Pro Val Leu | | |
| Glu Pro Leu Val Ala Val His Arg | | |
| Glu Ile Tyr Pro Lys Ala Asp Leu | | |
| Ser Ile Leu Gln Arg Ala Tyr Glu | | |
| Val Ala Asp Gln Arg His Ala Ser | | |
| Gln Leu Arg Gln Ser Gly Asp Pro | | |
| Tyr Ile Thr His Pro Leu Ala Val | | |
| Ala Asn Ile Leu Ala Glu Leu Gly | | |
| Met Asp Thr Thr Thr Leu Val Ala | | |
| Ala Leu Leu His Asp Thr Val Glu | | |
| Asp Thr Gly Tyr Thr Leu Glu Ala | | |
| Leu Thr Glu Glu Phe Gly Glu Glu | | |
| Val Gly His Leu Val Asp Gly Val | | |
| Thr Lys Leu Asp Arg Val Val Leu | | |
| Gly Ser Ala Ala Glu Gly Glu Thr | | |
| Ile Arg Lys Met Ile Thr Ala Met | | |
| Ala Arg Asp Pro Arg Val Leu Val | | |
| Ile Lys Val Ala Asp Arg Leu His | | |
| Asn Met Arg Thr Met Arg Phe Leu | | |
| Pro Pro Glu Lys Gln Ala Arg Lys | | |
| Ala Arg Glu Thr Leu Glu Val Ile | | |
| Ala Pro Leu Ala His Arg Leu Gly | | |
| Met Ala Ser Val Lys Trp Glu Leu | | |
| Glu Asp Leu Ser Phe Ala Ile Leu | | |
| His Pro Lys Lys Tyr Glu Glu Ile | | |
| Val Arg Leu Val Ala Gly Arg Ala | | |
| Pro Ser Arg Asp Thr Tyr Leu Ala | | |
| Lys Val Arg Ala Glu Ile Val Asn | | |
| Thr Leu Thr Ala Ser Lys Ile Lys | | |
| Ala Thr Val Glu Gly Arg Pro Lys | | |
| His Tyr Trp Ser Ile Tyr Gln Lys | | |
| Met Ile Val Lys Gly Arg Asp Phe | | |
| Asp Asp Ile His Asp Leu Val Gly | | |
| Val Arg Ile Leu Cys Asp Glu Ile | | |
| Arg Asp Cys Tyr Ala Ala Val Gly | | |
| Val Val His Ser Leu Trp Gln Pro | | |
| Met Ala Gly Arg Phe Lys Asp Tyr | | |
| Ile Ala Gln Pro Arg Tyr Gly Val | | |
| Tyr Gln Ser Leu His Thr Thr Val | | |
| Val Gly Pro Glu Gly Lys Pro Leu | | |
| Glu Val Gln Ile Arg Thr Arg Asp | | |
| Met His Arg Thr Ala Glu Tyr Gly | | |
| Ile Ala Ala His Trp Arg Tyr Lys | | |
| Glu Ala Lys Gly Arg Asn Gly Val | | |
| Leu His Pro His Ala Ala Ala Glu | | |
| Ile Asp Asp Met Ala Trp Met Arg | | |
| Gln Leu Leu Asp Trp Gln Arg Glu | | |
| Ala Ala Asp Pro Gly Glu Phe Leu | | |
| Glu Ser Leu Arg Tyr Asp Leu Ala | | |
| Val Gln Glu Ile Phe Val Phe Thr | | |
| Pro Lys Gly Asp Val Ile Thr Leu | | |
| Pro Thr Gly Ser Thr Pro Val Asp | | |
| Phe Ala Tyr Ala Val His Thr Glu | | |
| Val Gly His Arg Cys Ile Gly Ala | | |
| Arg Val Asn Gly Arg Leu Val Ala | | |
| Leu Glu Arg Lys Leu Glu Asn Gly | | |
| Glu Val Val Glu Val Phe Thr Ser | | |
| Lys Ala Pro Asn Ala Gly Pro Ser | | |
| Arg Asp Trp Gln Gln Phe Val Val | | |
| Ser Pro Arg Ala Lys Thr Lys Ile | | |
| Arg Gln Trp Phe Ala Lys Glu Arg | | |
| Arg Glu Glu Ala Leu Glu Thr Gly | | |
| Lys Asp Ala Met Ala Arg Glu Val | | |
| Arg Arg Gly Gly Leu Pro Leu Gln | | |
| Arg Leu Val Asn Gly Glu Ser Met | | |
| Ala Ala Val Ala Arg Glu Leu His | | |
| Tyr Ala Asp Val Ser Ala Leu Tyr | | |
| Thr Ala Ile Gly Glu Gly His Val | | |
| Ser Ala Lys His Val Val Gln Arg | | |
| Leu Leu Ala Glu Leu Gly Gly Ile | | |
| Asp Gln Ala Glu Glu Glu Leu Ala | | |
| Glu Arg Ser Thr Pro Ala Thr Met | | |
| Pro Arg Arg Pro Arg Ser Thr Asp | | |
| Asp Val Gly Val Ser Val Pro Gly | | |
| Ala Pro Gly Val Leu Thr Lys Leu | | |
| Ala Lys Cys Cys Thr Pro Val Pro | | |
| Gly Asp Val Ile Met Gly Phe Val | | |
| Thr Arg Gly Gly Gly Val Ser Val | | |
| His Arg Thr Asp Cys Thr Asn Ala | | |
| Ala Ser Leu Gln Gln Gln Ala Glu | | |
| Arg Ile Ile Glu Val Leu Trp Ala | | |
| Pro Ser Pro Ser Ser Val Phe Leu | | |
| Val Ala Ile Gln Val Glu Ala Leu | | |
| Asp Arg His Arg Leu Leu Ser Asp | | |
| Val Thr Arg Ala Leu Ala Asp Glu | | |
| Lys Val Asn Ile Leu Ser Ala Ser | | |
| Val Thr Thr Ser Gly Asp Arg Val | | |
| Ala Ile Ser Arg Phe Thr Phe Glu | | |
| Met Gly Asp Pro Lys His Leu Gly | | |
| His Leu Leu Asn Ala Val Arg Asn | | |
| Val Glu Gly Val Tyr Asp Val Tyr | | |
| Arg Val Thr Ser Ala Ala | | |

In some embodiments, the present disclosure provides a synthetic polypeptide molecule comprising at least one of the polypeptides described herein along with at least one other polypeptide. The other polypeptide can exist as a separate polypeptide of the fusion protein, or can exist as a polypeptide, which is expressed in frame (in tandem) with one of the polypeptides described herein. The other polypeptide can encode any peptidic or proteinaceous molecule, or a portion thereof. Suitable methods of making fusion proteins are known in the art, and include, for example, recombinant methods. See, for instance, Choi et al., Mol. Biotechnol. 31:193-202 (2005), which is incorporated by reference.

Included in the scope of the present disclosure are functional variants of the fusion proteins, and polypeptides, and proteins described herein. Functional variants can include fusion proteins, polypeptides, or proteins having substantial or significant sequence identity or similarity to a parent fusion proteins, polypeptides, or proteins, which functional variant retains the biological activity of the fusion proteins, polypeptides, or proteins of which it is a variant. In reference to the parent fusion proteins, polypeptides, or proteins, the functional variant can, for instance, be at least about 30%, 50%, 75%, 80%, 90%, 98% or more identical in amino acid sequence to the parent fusion proteins, polypeptide, or protein.

The functional variant can, for example, comprise the amino acid sequence of the parent fusion proteins, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent fusion proteins, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent fusion proteins, polypeptide, or protein.

The fusion polypeptides, and/or proteins of the invention (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; and U.S. Pat. No. 5,449,752, which are each incorporated by reference. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, NY 2001; and Ausubel et al., Current Protocols in Molecular Biology, Greene Publishing Associates and John Wiley & Sons, NY, 1994. Further, some of the fusion proteins, polypeptides, and proteins of the present disclosure (including functional portions and functional variants thereof) can be isolated and/or purified from a source, such as a plant, a bacterium, an insect, a mammal, e.g., a rat, a mouse, a human, etc. Methods of isolation and purification are well-known in the art. Alternatively, the fusion proteins, polypeptides, and/or proteins described herein (including functional portions and functional variants thereof) can be commercially synthesized by companies, such as Synpep (Dublin, CA), Peptide Technologies Corp. (Gaithersburg, MD), and Multiple Peptide Systems (San Diego, CA). In this respect, the fusion proteins, polypeptides, and proteins can be synthetic, recombinant, isolated, and/or purified.

In accordance with some embodiments, the present disclosure provides various pharmaceutical compositions comprising the DNA constructs or polypeptide compositions described herein for use as a vaccine. Thus, in further embodiments, the present disclosure provides the use of a pharmaceutical composition comprising a vaccine, and a pharmaceutically acceptable carrier, as a medicament, preferably as a medicament for the treatment of a Mtb infection in a subject.

In a further embodiment, the present invention provides a method for treating an Mtb infection in a subject in need thereof comprising administering to the subject an effective amount of the nucleic acid vaccine compositions and/or the synthetic polypeptide compositions described herein.

In some embodiments, the present disclosure provides methods of providing prophylaxis to, and/or treating an Mtb infection in, a subject in need thereof comprising administering to the subject an effective amount of a composition disclosed herein. In some embodiments, the composition is administered to the subject prior to, concurrent with, and/or after administering at least one antibiotic agent to the subject. In some embodiments, the composition is administered as one or more boost doses after an initial administration of the composition to the subject.

In some embodiments, the term “administering” means that the compositions of the present disclosure are introduced into a subject, preferably a subject receiving treatment for an Mtb infection, and the compounds are allowed to come in contact with the one or more infected cells or population of cells in vivo. In some embodiments, the composition is administered intramuscularly and/or intranasally to the subject.

It will be understood to persons having ordinary skill in the art that the nucleic acid vaccine or polypeptide vaccine compositions described herein can be administered in a regimen where there is a first or priming dose of vaccine composition administered to the subject, then after a period of time (e.g., 5 to 180 or more days), a second, third or more boost dose of vaccine is then administered to the subject. In some embodiments, the boost dose is administered 5, 6, 7, 8, 9, 10, 15, 20, 30, 40 up to 50 days apart.

The carrier is preferably a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well known to those skilled in the art and are readily available to the public. It is preferred that the pharmaceutically acceptable carrier be one which is chemically inert to the active agent(s) and one which has no detrimental side effects or toxicity under the conditions of use. In some embodiments, the pharmaceutical compositions of the present disclosure further include at least one additional biologically active agent (e.g., an antibiotic agent or the like).

The choice of carrier will be determined in part by the chemical properties of the vaccines as well as by the particular method used to administer the vaccines. Accordingly, there are a variety of suitable formulations of the pharmaceutical composition of the invention. The following formulations for intranasal, parenteral, subcutaneous, intravenous, intramuscular, intradermal, intraarterial, intrathecal and intraperitoneal administration are exemplary and are in no way limiting. More than one route can be used to administer the first and second vaccine, and in certain instances, a particular route can provide an immediate and more effective response than another route.

Injectable formulations are in accordance with the present invention. Formulations for effective pharmaceutical carriers for injectable compositions are well-known to persons having ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 14th ed., (2007)).

In accordance with some embodiments, the vaccines of the present invention can be administered other ways known in the art. For example, the vaccines can be administered via use of electroporation techniques. Suitable electroporation techniques are disclosed in U.S. Pat. Nos. 6,010,613, 6,603,998, and 6,713,291, all of which are incorporated herein by reference. Other physical approaches can include needle-free injection systems (NFIS) (e.g., as disclosed in U.S. Pat. No. 9,333,300, which is incorporated herein by reference), gene gun, biojector, ultrasound, and hydrodynamic delivery, all of which employ a physical force that permeates the cell membrane and facilitates intracellular gene transfer. Chemical vaccination approaches typically use synthetic or naturally occurring compounds (e.g., cationic lipids, cationic polymers, lipid-polymer hybrid systems) as carriers to deliver the nucleic acid into the cells.

In some other embodiments, intramuscular administration of the vaccines of the present invention may be achieved by the use of a needless injection device to administer a virus or plasmid DNA suspension (using, e.g., Biojector™) or a freeze-dried powder containing the vaccine (e.g., in accordance with techniques and products of Powderject).

In some aspects the vaccines disclosed herein are formulated in a lipid nanoparticle (LNP). The use of LNPs enables the effective delivery of chemically vaccines. Both modified and unmodified LNP formulated vaccines are optionally utilized. In some embodiments the vaccines disclosed herein are superior to conventional vaccines by a factor of at least 10 fold, 20 fold, 40 fold, 50 fold, 100 fold, 500 fold or 1,000 fold.

In one set of embodiments, lipid nanoparticles (LNPs) are provided. In one embodiment, a lipid nanoparticle comprises lipids including an ionizable lipid (such as an ionizable cationic lipid), a structural lipid, a phospholipid, and the nucleic acid vaccine. Each of the LNPs described herein or otherwise known to persons having ordinary skill in the art may be used as a formulation for the vaccines described herein. In some embodiments, the LNP comprises an ionizable lipid, a PEG-modified lipid, a phospholipid and a structural lipid. In some embodiments, the LNP has a molar ratio of about 20-60% ionizable lipid:about 5-25% phospholipid:about 25-55% structural lipid; and about 0.5-15% PEG-modified lipid. In some embodiments, the LNP comprises a molar ratio of about 50% ionizable lipid, about 1.5% PEG-modified lipid, about 38.5% structural lipid and about 10% phospholipid. In some embodiments, the LNP comprises a molar ratio of about 55% ionizable lipid, about 2.5% PEG lipid, about 32.5% structural lipid and about 10% phospholipid. In some embodiments, the ionizable lipid is an ionizable amino or cationic lipid and the phospholipid is a neutral lipid, and the structural lipid is a cholesterol. In some embodiments, the LNP has a molar ratio of 50:38.5:10:1.5 of ionizable lipid:cholesterol:DSPC:PEG2000-DMG. Additional details regarding LNPs and other carriers that are optionally adapted for use with the vaccines of the present disclosure are also described in, for example, U.S. Patent Application Publication No. US20200254086, which is incorporated by reference in its entirety.

For purposes of the present disclosure, the amount or dose of the vaccine administered should be sufficient to effect, e.g., a therapeutic or prophylactic response, in the subject over a selected time frame. The dose will typically be determined by the efficacy of the first and second vaccine and the condition of the given subject, as well as the body weight of that subject to be treated.

Typically, the attending physician will decide the dosage of first and second vaccine with which to treat each individual patient, taking into consideration a variety of factors, such as age, body weight, general health, diet, sex, to be administered, route of administration, and the severity of the condition being treated. By way of example and not intending to limit the invention, the dose of the vaccine is about 1 to 10,000 μg of vaccine to the subject being treated. In some embodiments, the dosage range of the vaccine is about 500 μg-6,000 μg of vaccine. In a preferred embodiment, the dosage of the vaccine is about 3,000 μg.

In accordance with some embodiments, the present disclosure provides pharmaceutical compositions comprising the nucleic acid vaccine compositions and/or the polypeptide compositions described herein in combination with at least one additional biologically active agent. An “active agent” and a “biologically active agent” are used interchangeably herein to refer to a chemical or biological compound that induces a desired pharmacological and/or physiological effect in which the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs, antibiotics, etc. The active agent can be a biological entity, such as a virus or cell, whether naturally occurring or manipulated, such as transformed.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

Non-limiting examples of biologically active agents include following: anti-inflammatory agents such as steroids, non-steroidal anti-inflammatory agents, anti-pyretic and analgesic agents, antigenic materials, antibiotics, and anti-viral drugs.

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

It will be understood that the methods of treatment using an effective amount of the nucleic acid vaccine compositions in combination with an effective amount of one or more additional biologically active agents, the combination can occur either simultaneously or serially with at least one other. The dosing regimens of the above methods can also comprise a first dose of vaccine an additional biologically active agent, followed by a second or more dose of vaccine and optionally an additional biologically active agent as needed.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In accordance with an embodiment, the present invention provides a cell or population of cells expressing the synthetic polypeptide compositions described herein. It will be understood that the cells or population of cells expressing the synthetic polypeptide compositions were in contact with the nucleic acid vaccine compositions and/or the synthetic polypeptide compositions in vitro or in vivo.

As defined herein, in another embodiment, the term “contacting” means that the one or more compounds of the present disclosure are introduced into a sample having at least one cell and appropriate enzymes or reagents, in a test tube, flask, tissue culture, chip, array, plate, microplate, capillary, or the like, and incubated at a temperature and time sufficient to permit binding and uptake of the at least one compound to the cell. Methods for contacting the samples with the compounds, and other specific binding components are known to those skilled in the art, and may be selected depending on the type of assay protocol to be run. Incubation methods are also standard and are known to those skilled in the art.

In accordance with an embodiment, the present disclosure provides kits that contain the compositions or pharmaceutical compositions used with the methods, as described above, to practice the methods of the invention. The kits can contain various combinations of vaccines and the like. The kit can contain instructional material teaching methodologies, e.g., means to administer the compositions used to practice the methods, means to inject or infect cells, patients or animals with vaccines of the present disclosure, means to monitor the resultant immune response and assess the reaction of the individual to which the compositions have been administered, and the like.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1: An Intranasal Stringent Response Vaccine Targeting Dendritic Cells as a Novel Adjunctive Therapy Against Tuberculosis

### Results

MIP-3α Fusion and IN Delivery of the relMtb Vaccine Individually Increase the Mycobactericidal Activity of INH in a Murine Model of Chronic TB

Four weeks after Mtb aerosol infection, C57BL/6 mice were treated daily with human-equivalent doses of oral INH for 10 weeks (FIG. 1B). The rel_{Mtb} or the fusion vaccine [FIG. 1A, detailed sequences are available in the Table 1] was administered via the IM [Standard Dose (SD) or High Dose (HD)] or IN (HD) routes weekly for 3 weeks. The original SD rel_{Mtb} vaccine given IM, which previously demonstrated therapeutic adjunctive activity together with INH, served as our baseline comparator in this study (“comparator” vaccine). For IN delivery, only the HD vaccine was tested to compensate for the anticipated reduced plasmid uptake without electroporation, which cannot be used with this vaccination route. One negative control group received no treatment, while another group received INH only. All vaccinated groups received INH in addition to the tested vaccines. Since DNA vaccination alone did not exhibit significant mycobactericidal activity in prior work, this group was not included in the present study.

At 10 weeks after primary vaccination by the IM route, greater potentiation of mycobactericidal activity of INH was observed in groups receiving the SD fusion vaccine compared to mice receiving the comparator vaccine [absolute reduction of mycobacterial burden: 0.63 log₁₀ colony-forming units (P=0.0001)]. Also, IN vaccination with the rel_{Mtb} vaccine significantly enhanced the mycobactericidal activity of INH compared to IM vaccination with the comparator vaccine [absolute reduction of mycobacterial burden: 0.52 log₁₀ colony-forming units (P=0.0052), FIG. 1C]. Interestingly, IM vaccination with the HD fusion or rel_{Mtb} vaccines did not significantly increase the mycobactericidal activity of INH relative to the comparator vaccine, indicating that the improved therapeutic efficacy observed with the IN vaccination approach is not simply dose-related. (FIG. 1C).

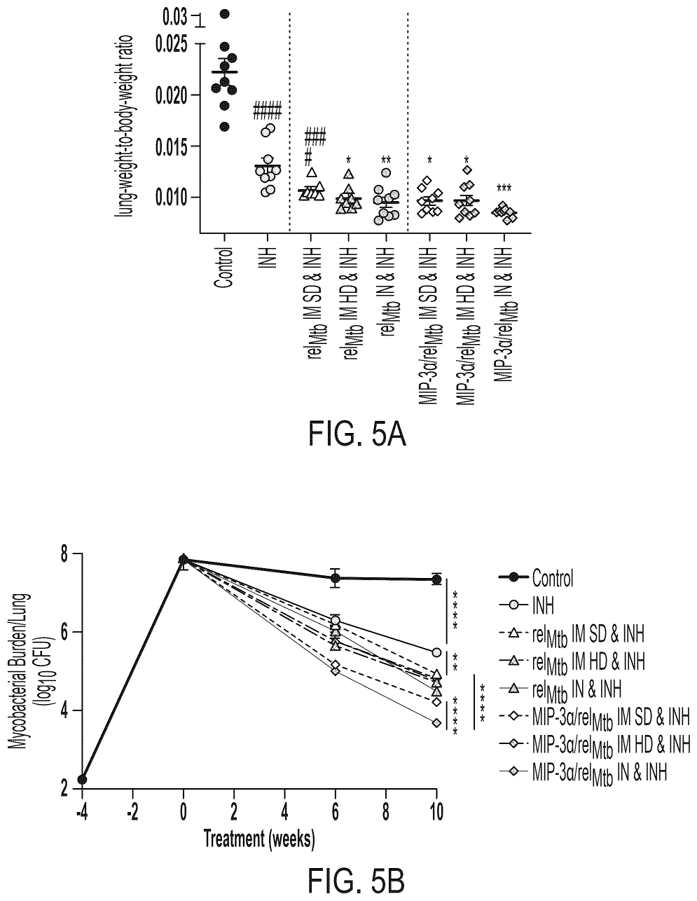

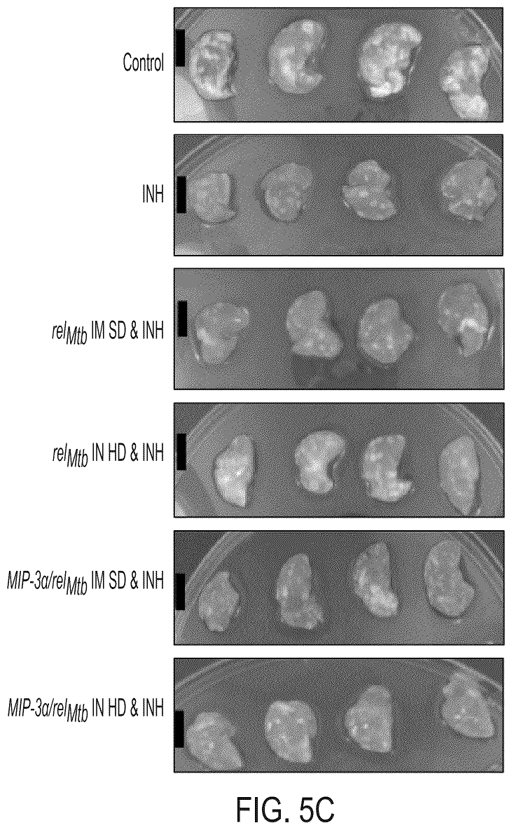

IN vaccination with the fusion vaccine (hereafter referred to as “optimized vaccination strategy”) showed the greatest additive therapeutic effect in combination with INH compared to any other experimental group; there was an absolute reduction in lung bacillary load of 1.13 log₁₀ relative to the comparator vaccine, of 0.5 log₁₀ relative to the IM-delivered SD fusion vaccine (P=0.0058; FIG. 1C), and of 0.61 log₁₀ relative to the IN-delivered rel_{Mtb} vaccine (P<0.0001; FIG. 1C). At 10 weeks post-primary vaccination, the optimized vaccination strategy resulted in the greatest reduction in normalized mean lung weight, which serves as a proxy for total lung inflammation, relative to the INH only group (relative reduction in normalized lung weight by 42.4%; P<0.0002) and untreated control group (relative reduction in normalized lung weight by 66.3%; P<0.0001) (FIG. 5A). All the individual comparisons between the different experimental groups are available in Table 5. Mean lung mycobacterial burdens at implantation (−4 weeks), initiation of treatment (0 weeks), and at 6 weeks and 10 weeks after the initiation of treatment for each experimental group are shown in FIG. 5B. Gross pathology photographs of representative lungs per experimental group are available in FIG. 5C.

Table 5. Lung mycobacterial burden of Mtb-infected mice at 10 weeks post infection. INH: Isoniazid, IM: Intramuscular, IN: Intranasal, SD: Standard dose, HD: High dose

LUNG MYCOBACTERIAL BURDEN AT 10 WEEKS

| | Adjusted |
|---|---|
| Comparisons | P Value |
| Control vs. INH | <0.0001 |
| Control vs. rel_{Mtb} IM SD & INH | <0.0001 |
| Control vs. rel_{Mtb} IM HD & INH | <0.0001 |
| Control vs. rel_{Mtb} IN HD & INH | <0.0001 |
| Control vs. MIP-3α/rel_{Mtb} IM SD & INH | <0.0001 |
| Control vs. MIP-3α/rel_{Mtb} IM HD & INH | <0.0001 |
| Control vs. MIP-3α/rel_{Mtb} IN HD & INH | <0.0001 |
| INH vs. rel_{Mtb} IM SD & INH | 0.0076 |
| INH vs. rel_{Mtb} IM HD & INH | <0.0001 |
| INH vs. rel_{Mtb} IN HD & INH | <0.0001 |
| INH vs. MIP-3α/rel_{Mtb} IM SD & INH | <0.0001 |
| INH vs. MIP-3α/rel_{Mtb} IM HD & INH | 0.0002 |
| INH vs. MIP-3α/rel_{Mtb} IN HD & INH | <0.0001 |
| rel_{Mtb} IM SD & INH vs. rel_{Mtb} IM HD & INH | 0.8952 |
| rel_{Mtb} IM SD & INH vs. rel_{Mtb} IN HD & INH | 0.0052 |
| rel_{Mtb} IM SD & INH vs. MIP-3α/rel_{Mtb} IM SD & INH | 0.0001 |
| rel_{Mtb} IM SD & INH vs. MIP-3α/rel_{Mtb} IM HD & INH | 0.9901 |
| rel_{Mtb} IM SD & INH vs. MIP-3α/rel_{Mtb} IN HD & INH | <0.0001 |
| rel_{Mtb} IM HD & INH vs. rel_{Mtb} IN HD & INH | 0.5415 |
| rel_{Mtb} IM HD & INH vs. MIP-3α/rel_{Mtb} IM SD & INH | 0.0050 |
| rel_{Mtb} IM HD & INH vs. MIP-3α/rel_{Mtb} IM HD & INH | 0.9997 |
| rel_{Mtb} IM HD & INH vs. MIP-3α/rel_{Mtb} IN HD & INH | <0.0001 |
| rel_{Mtb} IN HD & INH vs. MIP-3α/rel_{Mtb} IM SD & INH | 0.4858 |
| rel_{Mtb} IN HD & INH vs. MIP-3α/rel_{Mtb} IM HD & INH | 0.2519 |
| rel_{Mtb} IN HD & INH vs. MIP-3α/rel_{Mtb} IN HD & INH | <0.0001 |
| MIP-3α/rel_{Mtb} IM SD & INH vs. MIP-3α/rel_{Mtb} IM | 0.0010 |
| HD & INH | |
| MIP-3α/rel_{Mtb} IM SD & INH vs. MIP-3α/rel_{Mtb} IN | 0.0058 |
| HD & INH | |
| MIP-3α/rel_{Mtb} IM HD & INH vs. MIP-3α/rel_{Mtb} IN | <0.0001 |
| HD & INH | |

IM Vaccination with the Fusion Vaccine Elicits a Robust Systemic Th1 Response

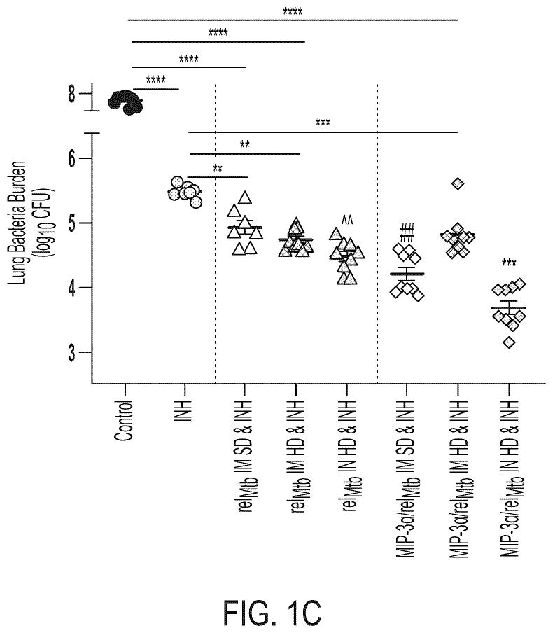

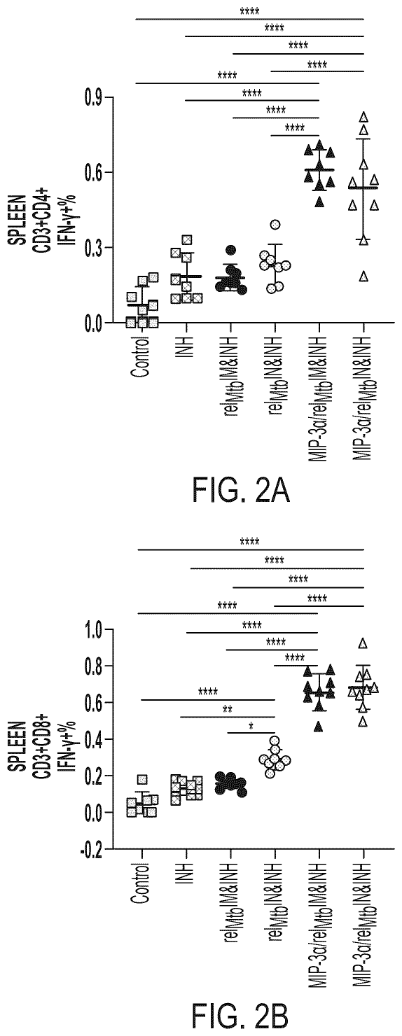

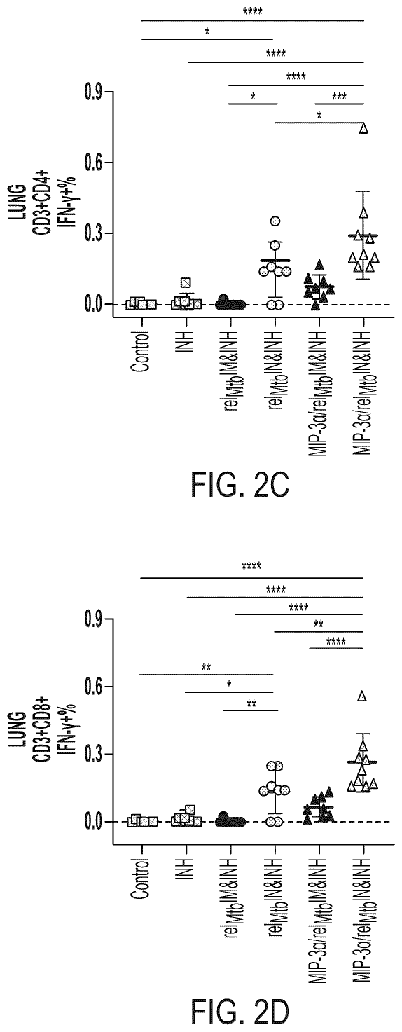

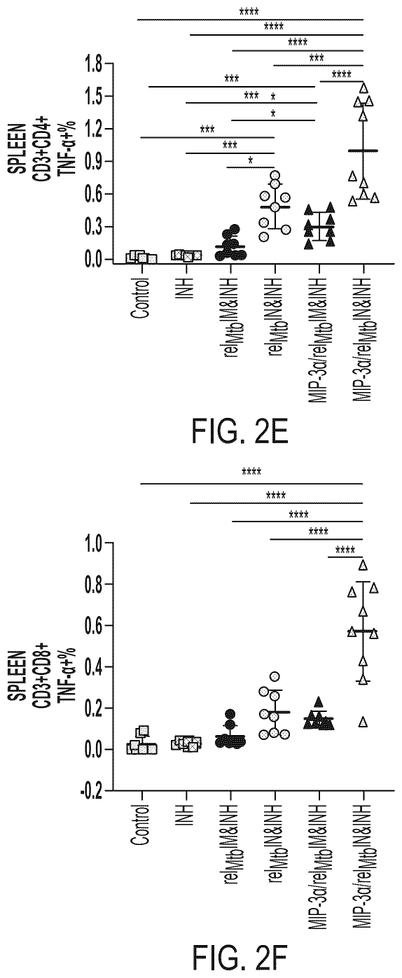

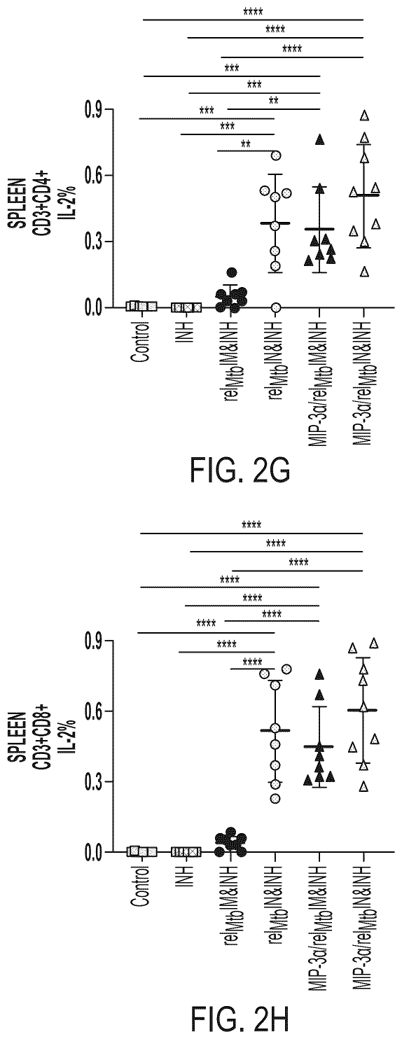

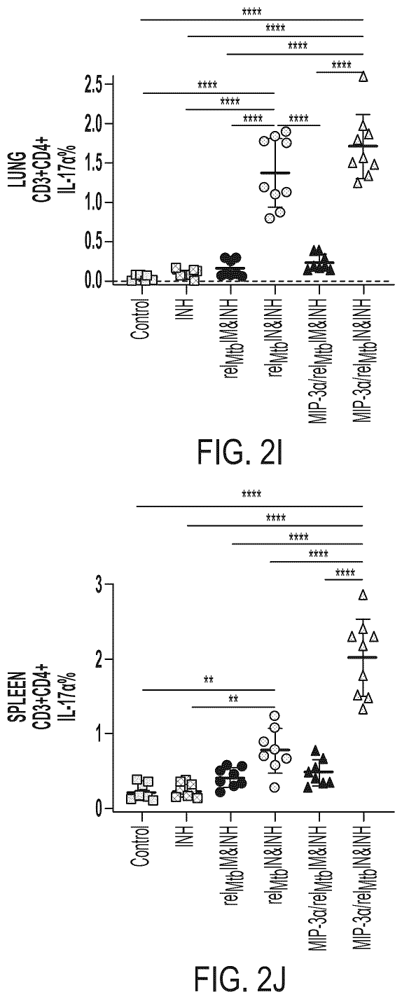

For simplicity, we have included only the SD IM vaccine groups and not the HD IM groups, since both IM groups yielded similar microbiological outcomes. Relative to the comparator vaccine, the IM fusion vaccine elicited substantially higher numbers of Rel_{Mtb}-specific, IFN-γ-producing CD4+ and CD8+T lymphocytes in the spleens (P<0.0001 and P<0.0001; FIGS. 2A and B), but not in the lungs of infected mice (FIGS. 2C and 2D). Relative to the comparator vaccine, the IM-delivered fusion vaccine was also associated with significantly increased numbers of Rel_{Mtb}-specific, TNF-α-producing CD4+ T cells (P=0.0076, FIG. 2E) but not CD8+ T cells (FIG. 2F) in the spleens of Mtb-infected mice. Significant production of Rel_{Mtb}-specific, IL-2-producing CD4+ and CD8+ T cells in the spleens of infected mice was also noted after IM vaccination with the fusion vaccine relative to the comparator vaccine (P=0.005 and P<0.0001, respectively, FIGS. 2G and 2H). IM vaccination with the fusion vaccine or the comparator vaccine elicited similar numbers of Rel_{Mtb}-specific, IL-17α-producing CD4+ T cells in infected murine lungs or spleens (FIGS. 21 and 2J). All the individual statistical comparisons of IM vaccination with the fusion vaccine relative to all the other experimental groups are available in Table 6.

Table 6. T-cell responses in Mtb-infected murine tissues 6 weeks post treatment initiation. INH: Isoniazid, IM: Intramuscular, IN: Intranasal.

T-cell responses in Mtb-infected murine tissues

| | SPLEEN | | | | | | | LUNG | | |
|---|---|---|---|---|---|---|---|---|---|---|
| | IFN-γ | | TNF- α | | IL-2 | | IL-17α | IFN-γ | | IL-17α |
| Turkey's Multiple | CD4 + T | CD8 + T | CD4 + T | CD8 + T | CD4 + T | CD8 + T | CD4 + T | CD4 + T | CD8 + T | CD4 + T |
| Comparisons Test | Adjusted P Value | | | | | | | | | |
| Control vs. INH | 0.3348 | 0.2939 | >0.9999 | >0.9999 | >0.9999 | >0.9999 | >0.9999 | 0.9999 | >0.9999 | 0.9990 |
| Control vs. rel_{Mtb} IM & INH | 0.3741 | 0.0838 | 0.9251 | 0.9815 | 0.9871 | 0.9949 | 0.7119 | >0.9999 | >0.9999 | 0.9616 |
| Control vs. rel_{Mtb} IN & INH | 0.0557 | <0.0001 | 0.0010 | 0.0972 | 0.0002 | <0.0001 | 0.0025 | 0.0447 | 0.0057 | <0.0001 |
| Control vs. MIP-3α/rel_{Mtb} IM | <0.0001 | <0.0001 | 0.0002 | 0.2972 | 0.0007 | <0.0001 | 0.4208 | 0.6696 | 0.4597 | 0.7090 |
| & INH | | | | | | | | | | |
| Control vs. MIP-3α/rel_{Mtb} IN | <0.0001 | <0.0001 | <0.0001 | <0.0001 | <0.0001 | <0.0001 | <0.0001 | <0.0001 | <0.0001 | <0.0001 |
| & INH | | | | | | | | | | |
| INH vs. rel_{Mtb} IM & INH | >0.9999 | 0.9876 | 0.9751 | 0.9894 | 0.9856 | 0.9941 | 0.7898 | 0.9998 | >0.9999 | 0.9975 |
| INH vs. rel_{Mtb} IN & INH | 0.9465 | 0.0020 | 0.0020 | 0.1159 | 0.0002 | <0.0001 | 0.0037 | 0.0805 | 0.0105 | <0.0001 |
| INH vs. MIP-3α/rel_{Mtb} IM & | <0.0001 | <0.0001 | 0.0002 | 0.3386 | 0.0007 | <0.0001 | 0.5046 | 0.8121 | 0.5964 | 0.8957 |
| INH | | | | | | | | | | |
| INH vs. MIP-3α/rel_{Mtb} IN & | <0.0001 | <0.0001 | <0.0001 | <0.0001 | <0.0001 | <0.0001 | <0.0001 | <0.0001 | <0.0001 | <0.0001 |
| INH | | | | | | | | | | |
| rel_{Mtb} IM & INH vs. rel_{Mtb} IN | 0.9268 | 0.0123 | 0.0167 | 0.3597 | 0.0018 | <0.0001 | 0.1052 | 0.0439 | 0.0063 | <0.0001 |
| & INH | | | | | | | | | | |
| rel_{Mtb} IM & INH vs. MIP-3α/ | <0.0001 | <0.0001 | 0.0076 | 0.7128 | 0.0050 | <0.0001 | 0.9970 | 0.6648 | 0.4816 | 0.9900 |
| rel_{Mtb} IM & INH | | | | | | | | | | |
| rel_{Mtb} IM & INH vs. MIP-3α/ | <0.0001 | <0.0001 | <0.0001 | <0.0001 | <0.0001 | <0.0001 | <0.0001 | <0.0001 | <0.0001 | <0.0001 |
| rel_{Mtb} IN & INH | | | | | | | | | | |
| rel_{Mtb} IN & INH vs. MIP-3α/ | <0.0001 | <0.0001 | 0.5341 | 0.9921 | 0.9993 | 0.9451 | 0.2627 | 0.6451 | 0.3761 | <0.0001 |
| rel_{Mtb} IM & INH | | | | | | | | | | |
| rel_{Mtb} IN & INH vs. MIP-3α/ | <0.0001 | <0.0001 | 0.0003 | <0.0001 | 0.5818 | 0.8022 | <0.0001 | 0.0340 | 0.0064 | 0.1005 |
| rel_{Mtb} IN & INH | | | | | | | | | | |
| MIP-3α/rel_{Mtb} IM & INH vs. | 0.717 | 0.9929 | <0.0001 | <0.0001 | 0.3635 | 0.2645 | <0.0001 | 0.0003 | <0.0001 | <0.0001 |
| MIP-3α/rel_{Mtb} IN & INH | | | | | | | | | | |

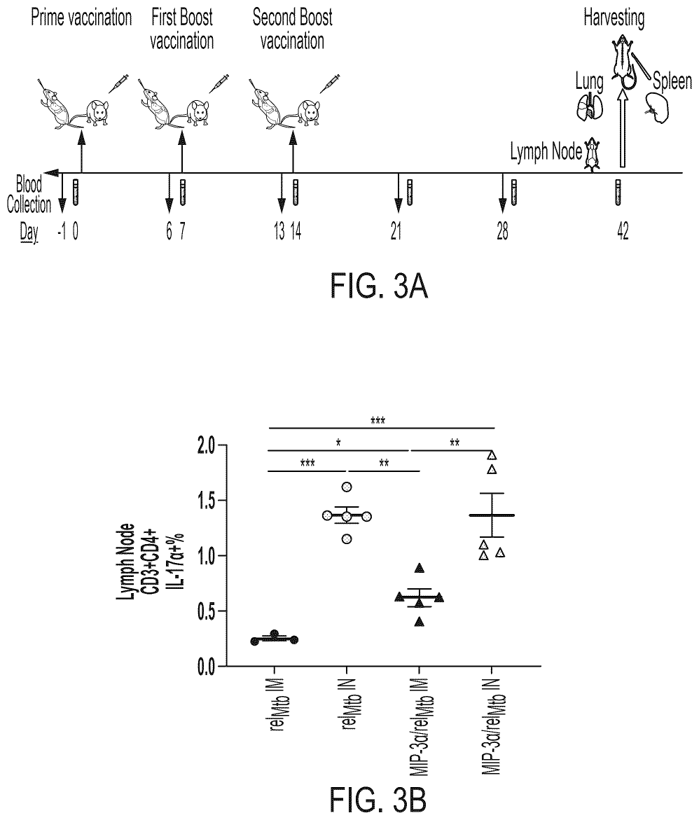

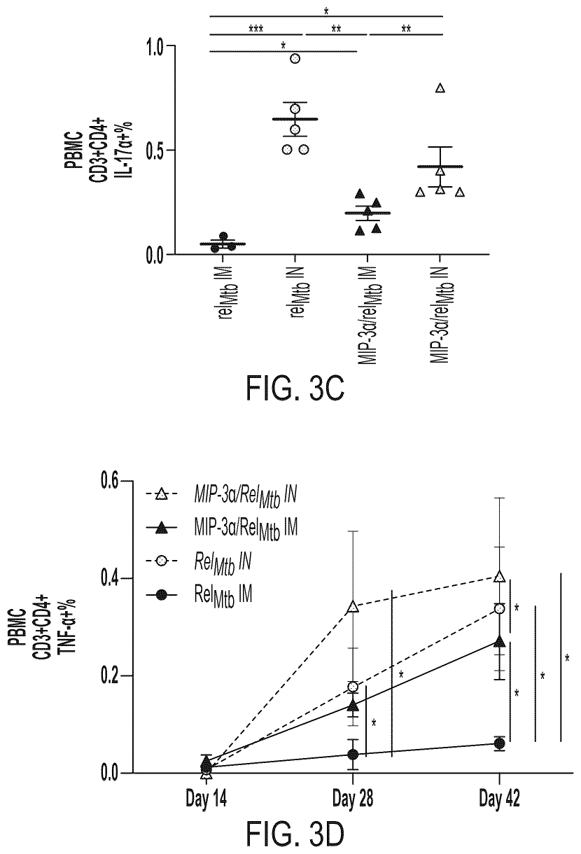

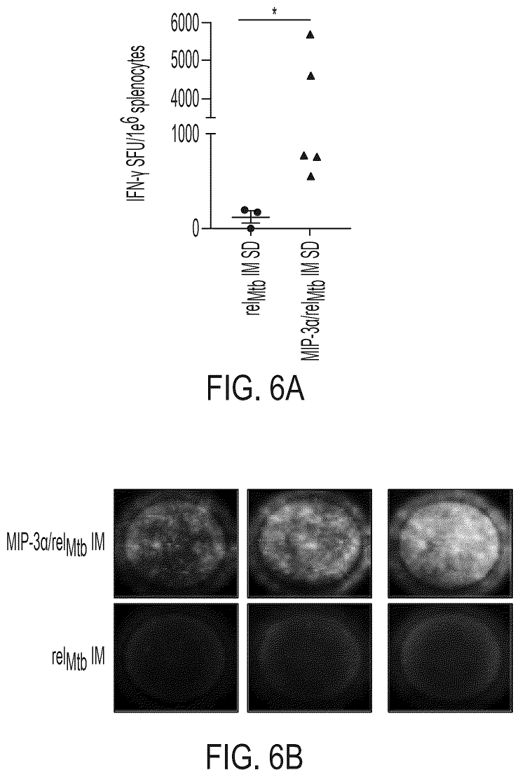

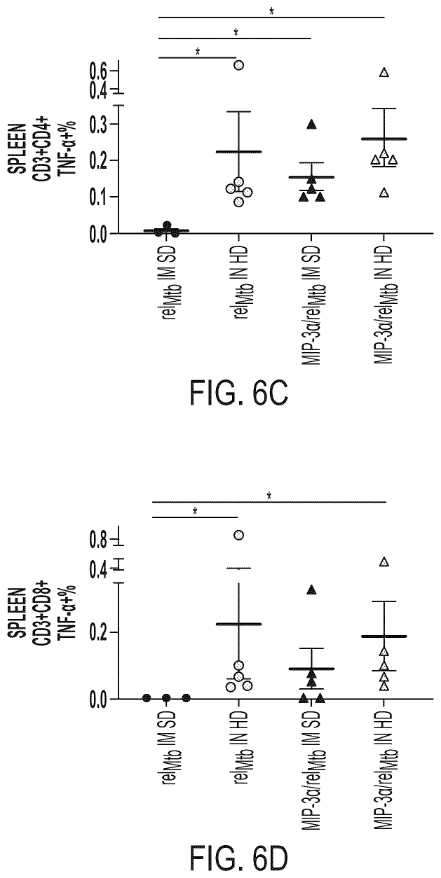

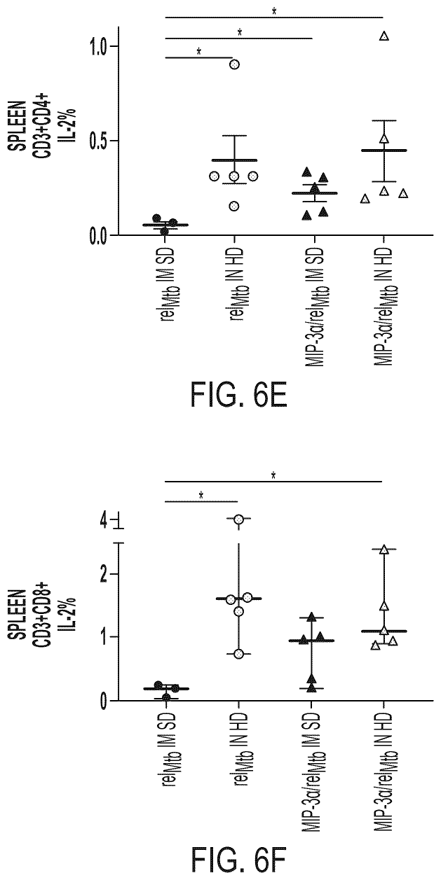

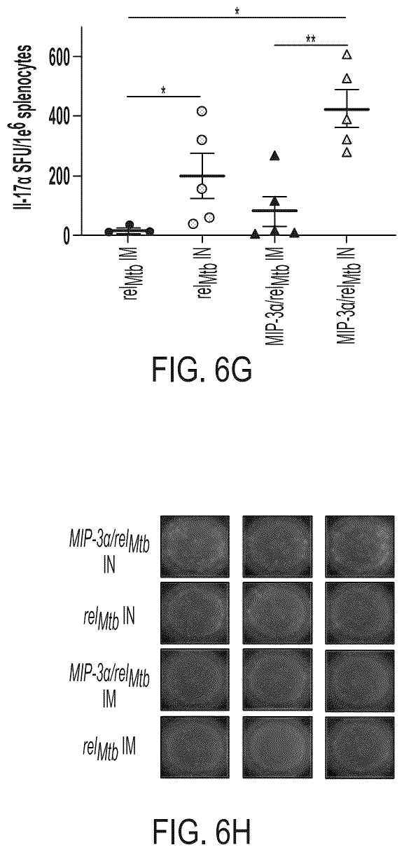

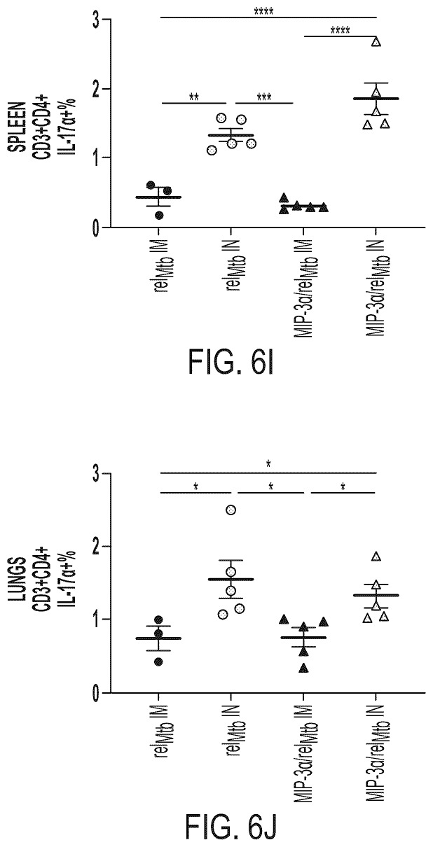

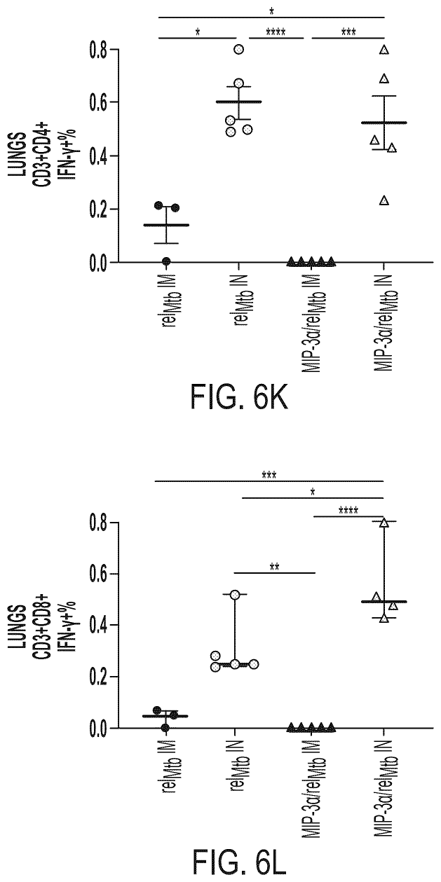

In an independent immunogenicity study using uninfected animals (FIG. 3A), we also tested the T-cell responses in additional murine tissues, including draining lymph nodes (LNs) (FIG. 3B) and peripheral blood mononuclear cells (PBMCs) (FIGS. 3C and 3D), in addition to spleens and lungs (FIG. 6). Relative to the comparator, IM vaccination with the fusion vaccine elicited more Rel_{Mtb}-specific, IL-17α-producing CD4+ T cells in the draining LNs and PBMCs (P=0.036 and P=0.036 respectively, FIGS. 3B and 3C). Similarly, IM delivery of the fusion vaccine elicited more Rel_{Mtb}-specific, TNF-α-producing CD4+ T cells in the PBMC population starting at 28 days (P=0.036) and peaking at 42 days (P=0.036) after primary vaccination (FIG. 3D). All the individual statistical comparisons of IM-delivered fusion vaccine with all the other experimental groups in uninfected animals are available in Table 7.

Table 7. T-cell responses in uninfected murine tissues 6 weeks after primary vaccination. IM: Intramuscular, IN: Intranasal. PBMC: peripheral blood mononuclear cells.

T-cell responses in uninfected murine tissues

| | Draining | | | |
|---|---|---|---|---|
| | Lymph | | | |
| | Nodes | PBMCs | PBMCs | |
| | IL-17α | IL-17α | TNF-α | |
| | CD4+ T | CD4+ T | CD4+ T | |
| Tukey's Multiple | | | Day 28 | Day 42 |
| Comparisons Test | | | | |
| | Adjusted P Value | | | |
| rel_{Mtb} IM vs. rel_{Mtb} IN | 0.0003 | 0.0007 | 0.085 | 0.036 |
| rel_{Mtb} IM vs. MIP-3α/rel_{Mtb} IM | 0.0357 | 0.0357 | 0.036 | 0.036 |
| rel_{Mtb} IM vs. MIP-3α/rel_{Mtb} IN | 0.0003 | 0.0265 | 0.025 | 0.010 |
| rel_{Mtb} IN vs. MIP-3α/rel_{Mtb} IM | 0.0033 | 0.0023 | 0.9889 | 0.9339 |
| rel_{Mtb} IN vs. MIP-3α/rel_{Mtb} IN | >0.9999 | 0.1522 | 0.5171 | 0.9462 |
| MIP-3α/rel_{Mtb} IM vs. | 0.0034 | 0.0079 | 0.123 | 0.036 |
| MIP-3α/rel_{Mtb} IN | | | | |

IN Vaccination with MIP-3α/Rel_{Mtb}, the Optimized Vaccination Strategy, Elicits the Most Robust Systemic and Local Th1/Th17 Responses Relative to any Other Vaccination Strategy

Having established that IM vaccination with the fusion vaccine induces enhanced systemic Th1 responses relative to the comparator, we next assessed the effect of the IN vaccination route on immune responses elicited by the fusion vaccine. The optimized vaccination strategy group had greater numbers of Rel_{Mtb}-specific, IFN-γ-producing CD4+ and CD8+ T cells in the lungs of Mtb-infected mice (P=0.003 and P<0.0001, FIGS. 2C and 2D, respectively) compared to the IM-delivered fusion vaccine. The optimized vaccination strategy also induced higher numbers of Rel_{Mtb}-specific, TNF-α-producing CD4+ and CD8+ T cells in the spleens compared to the IM-delivered fusion vaccine (P<0.0001 and P<0.0001, FIGS. 2E and 2F, respectively). Rel_{Mtb}-specific, IL-2-producing CD4+ and CD8+ T cells in the spleens of infected mice were similarly high irrespective of the route of delivery of the fusion vaccine (FIGS. 2G and 2H). Infected mice receiving the optimized vaccination strategy showed a significantly higher percentage of Rel_{Mtb}-specific, IL-17α-producing CD4+ T cells in the lungs and spleens compared to those receiving the fusion vaccine by the IM route (P<0.0001 and P<0.0001, FIGS. 21 and 2J).

Relative to IN vaccination with the rel_{Mtb} vaccine, the optimized vaccination strategy elicited greater numbers of Rel_{Mtb}-specific, IFN-γ-producing CD4+ and CD8+ T cells in the spleens (P<0.0001 and P<0.0001, FIGS. 2A and 2B, respectively) and in the lungs (P=0.034 and P=0.0064, FIGS. 2C and 2D, respectively) of infected mice. The optimized vaccination strategy also resulted in higher numbers of Rel_{Mtb}-specific, TNF-α-producing CD4+ and CD8+ T cells in the spleens compared to mice receiving the rel_{Mtb} vaccine (P=0.0003 and P<0.0001, FIGS. 2E and 2F, respectively). Rel_{Mtb}-specific IL-2-producing CD4+ and CD8+T lymphocytes in the spleens were similarly high in the optimized vaccination group and the IN rel_{Mtb} group (FIGS. 2G and 2H). Mice receiving the optimized vaccination strategy showed a significantly higher percentage of Rel_{Mtb}-specific, IL-17α-producing CD4+ T cells in the spleens compared to mice receiving the rel_{Mtb} vaccine by the IN route (P<0.0001, FIG. 2J), but no difference between the two groups was noted in the lungs (FIG. 21). Of note, no IL-17α response was observed at these sites following IM immunization.

Focusing on the differences in the immune responses in the IN rel_{Mtb} and the comparator groups, the former approach induced a significantly increased proportion of Rel_{Mtb}-specific, IFN-γ-producing CD4+ and CD8+ T cells in the lungs (P=0.044 and P=0.006; FIGS. 2C and D), and of IFN-γ-producing CD8+ T cells in the spleens of infected mice (P=0.012, FIG. 2B). IN vaccination with the rel_{Mtb} vaccine also induced significantly higher numbers of Rel_{Mtb}-specific, TNF-α-producing CD4+ T cells in the spleens relative to the comparator group (P=0.017, FIG. 2C). Moreover, relative to the comparator, IN vaccination with the rel_{Mtb} vaccine significantly increased Rel_{Mtb}-specific, IL-2-producing CD4+ and CD8+ T cells in the spleens [P=0.0018 and P<0.001, FIGS. 2G and H), and a higher percentage of Rel_{Mtb}-specific, IL-17α-producing CD4+ T cells in the lungs (P<0.0001; FIG. 21), but not in the spleens (FIG. 2J). All the individual statistical comparisons between experimental groups are available in Table 6.

In an independent immunogenicity study using uninfected animals (FIG. 3A), we found that, compared to fusion vaccine given IM, the optimized vaccination strategy elicited more robust Rel_{Mtb}-specific, IL-17α CD4+ T-cell responses in the draining LNs (P=0.0033, FIGS. 3B) and higher Rel_{Mtb}-specific, IL-17α CD4+ T-cell responses in PBMCs (P=0.0079, FIG. 3C). The optimized vaccination strategy also induced a higher percentage of Rel_{Mtb}-specific, TNF-α-producing CD4+ T cells at 42 days compared to the fusion vaccine given IM (P=0.03, FIG. 3D). IN vaccination with the rel_{Mtb} vaccine elicited a higher percentage of rel_{Mtb}-specific, IL-17α-producing CD4+ T cells relative to the comparator in the draining LNs (P=00003, FIG. 3B) and PBMCs (P=0.0007, FIG. 3C). Finally, by day 42 after primary vaccination, IN delivery of the rel_{Mtb} vaccine induced higher proportions of rel_{Mtb}-specific, TNF-α-producing CD4+ T cells in PBMCs relative to the comparator (P=0.036, FIG. 3D). The individual statistical comparisons of immunological outcomes in uninfected mice resulting from different vaccination protocols are available in Table 7.

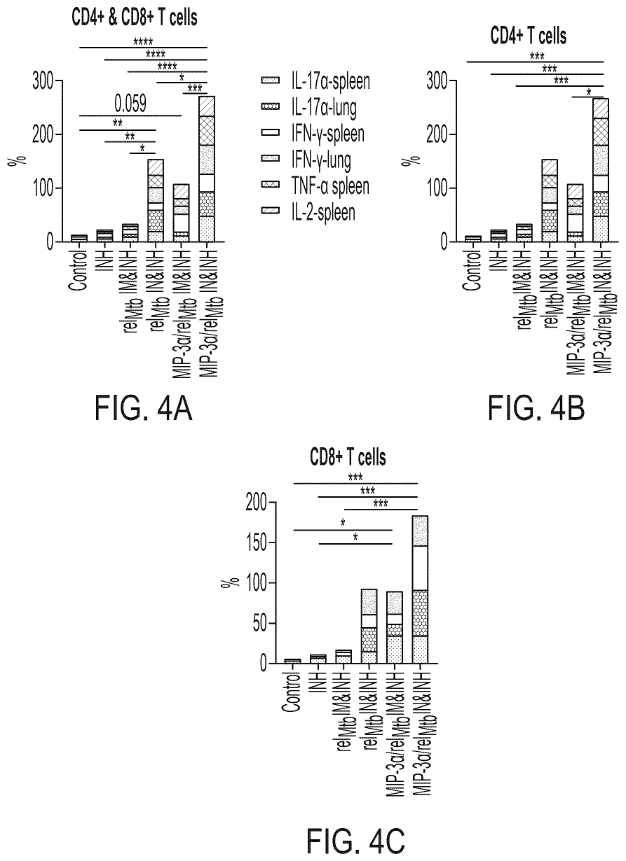

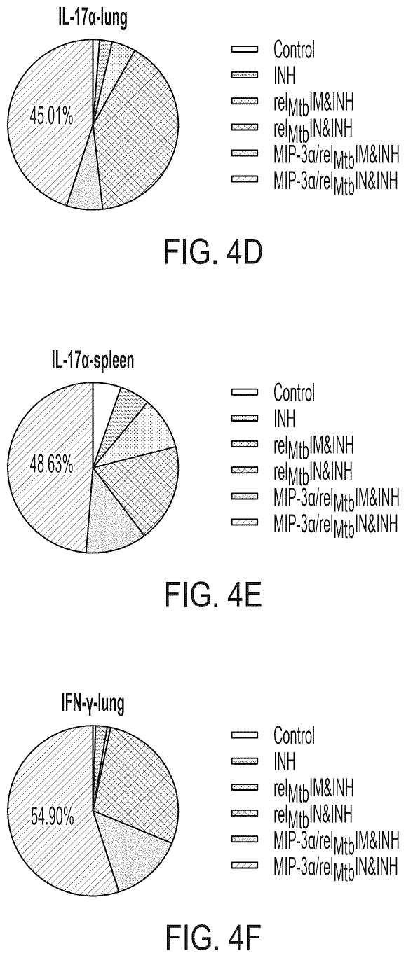

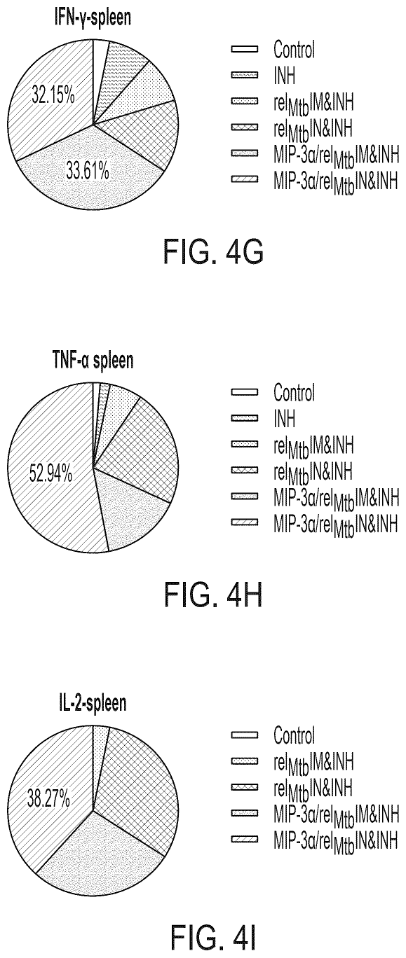

Collectively, the optimized vaccination strategy, which was shown to have the most favorable microbiological outcomes over any other tested vaccination approach (FIG. 1C), was also found, in parallel, to significantly increase the production of multiple cytokines associated with Mtb control, both systemically and at the site of infection. More specifically, compared to any other approach, the optimized vaccination strategy induced the highest numbers of rel_{Mtb}-specific CD4+ and CD8+ T cells producing IL17-α, TNF-α, IFN-γ, and IL—in the spleens and lungs of Mtb-infected animals (FIG. 4A, 3B, 3C, Table 6), as well as the highest normalized production of each individual cytokine (FIGS. 4D, 4E, 4F, 4G, 4H, 4I).

### DISCUSSION

The development of novel immunotherapeutic regimens that synergize with antibiotics to accelerate curative TB treatment is an attractive strategy for improving medical adherence and treatment completion rates, and for reducing costs. In the current study, we show that MIP-3α fusion and the IN route of delivery individually enhance the therapeutic adjunctive activity of a DNA vaccine targeting an Mtb persistence antigen in a murine model of chronic TB. Importantly, the combined approach, i.e., IN immunization with a DNA fusion vaccine expressing MIP-3α/rel_{Mtb}, was accompanied by additive Th1/Th17 responses, both systemically and at the site of infection. This novel optimized vaccination strategy may be a promising adjunctive therapeutic approach in combination with standard anti-TB therapy.

A true functional immunological signature to predict adequate TB control is still lacking, but it is clear that CD4+ and CD8+ T cells are critical in developing immunity against Mtb. T-cell immunity to TB is likely mediated by a variety of T cells, especially those mediating Th1 and Th1/Th17-like responses. Chronic antigenic stimulation drives antigen-specific CD4+ T-cell functional exhaustion during murine Mtb infection, with important implications for TB vaccine design. Thus, subdominant Mtb antigens during chronic Mtb infection, including Rel_{Mtb}, which is induced during antitubercular treatment, may represent promising targets for therapeutic vaccines in an effort to “re-educate” the immune system to tailor host anti-TB responses. In the current study, we focused on improving Rel_{Mtb}-specific T-cell responses by enhancing the engagement of immature DCs.

Immature DCs are critical for the activation of adaptive immunity, and, eventually, mature DCs trigger antigen-specific naïve T cells. Of note, only a small minority of DCs are attracted to sites of immunization, and, in the case of HIV and TB infections, a proportion of the attracted DCs may be dysfunctional. Fusion of the antigen of interest to the chemokine MIP-3α (or CCL20) targets the antigen to immature DCs. It has been shown that following naked DNA vaccination, epidermal cells secrete the antigen of interest-chemokine MIP-3α fusion construct. The secreted fusion construct is taken up and internalized by skin Langerhans cells via the receptor for this chemokine, which is called CCR6. The complex is then processed and presented in draining LNs to elicit efficient cellular and humoral responses. Enhanced efficacy has been shown compared to antigen-only vaccines in various systems. In a mouse melanoma model, our group has demonstrated that IM immunization with a DNA vaccine containing a fusion of MIP-3α with the tumor antigen gene gp100/Trp2 elicited greater numbers of tumor antigen-specific T cells and offered greater therapeutic benefit compared to the cognate vaccine lacking the MIP-3α fusion. Importantly, MIP-3α has also been shown to play a key role in driving DC recruitment to the nasal mucosa. Indeed, we found that IM vaccination with the MIP-3α fusion construct conferred increased antigen-specific systemic Th1 responses (IFN-γ, TNF-α, Il-2 in the spleens and TNF-α in PBMCs), but also Th17 responses in the draining LNs and PBMCs, relative to the rel_{Mtb} construct alone. Interestingly, no Th1 or Th17 responses were observed in the lungs, the primary site of the infection. This fusion vaccination strategy, i.e., IM vaccination with MIP-3α/rel_{Mtb}, yielded improved microbiological outcomes when combined with INH compared to the non-fused rel_{Mtb} vaccine.

Compelling evidence suggests that protection against respiratory pathogens, such as Mtb, is dependent on the presence of pathogen-specific immune cells at the primary site of infection. Pre-clinical studies have shown that parenteral immunization with TB vaccines can drive robust antigen-specific T-cell responses in the periphery, but these cells are unable to rapidly enter the restricted lung mucosal compartments and largely fail to restrict Mtb replication. In contrast, respiratory mucosal immunization generates a long-lasting population of tissue-resident T cells expressing homing molecules to allow preferential migration and residence in the airway lumen and lung parenchyma. These immune cells generate a ‘first line of defense’ by establishing pathogen-specific immunity at the site of entry, providing markedly enhanced control against pulmonary Mtb infection. Importantly, after IN vaccination, these antigen-experienced, lung-resident, T-cells have been shown to produce IL-17α in addition to IFN-γ, expanding the known signature panel that may confer enhanced TB immunity. In the current study, we have shown for the first time that IN delivery of a vaccine (rel_{Mtb}) enhances the bactericidal activity of an antitubercular drug (INH) relative to IM delivery of the same vaccine. IN immunization resulted in more robust Th1 (IFN-γ, TNF-α, IL-2) and Th17 responses (IL-17α) systemically, but also in the lungs (IFN-γ and IL-17), the primary site of infection. Importantly, the combined approach of MIP-3α fusion and the IN route of immunization yielded the greatest additive adjunctive mycobactericidal activity with INH in murine lungs, resulting in an approximate 100-fold reduction in lung bacterial burden compared to INH alone. This immunization protocol was accompanied by the most robust, additive Th1 and Th17 responses, both systemically and in the lungs of Mtb-infected mice.

In conclusion, we have shown that IN immunization with a DNA vaccine expressing MIP-3α/rel_{Mtb} generates strong, additive Th1 and Th17 responses and significantly potentiates the mycobactericidal activity of the first-line drug, INH. Further studies are required to elucidate the relative importance of the different effector mechanisms elicited by this immunization strategy and to refine our understanding of the host-pathogen interactions that result in the improved therapeutic effects. Ultimately, the potential utility of this vaccination combination strategy must be evaluated as an adjunctive therapeutic intervention in shortening the duration of curative treatment for active TB.

### Methods

### Bacteria and Growth Conditions

Wild-type Mtb H37Rv was grown in Middlebrook 7H9 broth (Difco, Sparks, MD) supplemented with 10% oleic acid-albumindextrose-catalase (OADC, Difco), 0.2% glycerol, and 0.05% Tween-80 at 37° C. in a roller bottle.

### Antigen Preparation

The previously generated rely expression plasmid, pET15b[rel_{Mtb}], was used for expression and purification of recombinant Rel_{Mtb} protein. Escherichia coli BL21 (DE3) RP competent cells (Stratagene) were transformed with pET15b[rel_{Mtb}]. Transformed bacteria were selected with ampicillin (100 mg/ml), and cloning was confirmed by DNA sequencing. Protein expression was performed using standard protocols and purification was performed using Ni-NTA Agarose (Qiagen). The Relat protein (87 kDa) was purified from the cell lysate using a Ni-NTA resin column. The purity was confirmed by SDS-PAGE gel and immunoblot analyses. The protein concentration was determined using a BCA protein assay with BSA as the standard (Thermo Fisher). Recombinant Rel_{Mtb} protein has been shown previously to retain (p)ppGpp synthesis and hydrolysis activities and has been used as an antigen to measure Rel_{Mtb}-specific T-cell responses ex vivo.

### DNA Vaccines

The plasmid pSectag2B encoding the full-length rel_{Mtb} gene was used as the rel_{Mtb} DNA vaccine. The rel_{Mtb} gene was codon-optimized (Genscript) and fused to the mouse MIP-3α gene. The fusion product was cloned into pSectag2B, serving as the MIP-3α/rel_{Mtb} DNA, fusion, vaccine or (FIG. 1A). Proper insertion was confirmed by sequencing and the expression of target genes was confirmed by transfection of 293T cells in lysates and supernatants. Vaccination plasmids were selected by ampicillin (100 μg/ml) and extracted from E. coli DH5-α (Invitrogen™ ThermoFisher Scientific, Waltham, MA) using Qiagen® (Germantown, MD) EndoFree® Plasmid Kits and were diluted with endotoxin-free 1×PBS.

### Mtb Challenge Study in Mice

Male and female C57BL/6 mice (8-10-week-old, The Jackson Laboratory) were aerosol-infected with ˜100 bacilli of wild-type Mtb H37Rv using a Glas-Col Inhalation Exposure System (Terre Haute, IN). After 28 days of infection, the mice received INH 10 mg/kg dissolved in 100 ml of distilled water by esophageal gavage once daily (5 days/week) and were randomized to receive rel_{Mtb} or the fusion vaccine by the intramuscular (standard dose: 20 μg or high dose: 200 μg) or intranasal (high dose: 200 μg) route. The mice were vaccinated three times at one-week intervals. Each plasmid was delivered IM or IN after mice were adequately anesthetized by vaporized isoflurane. For IM immunizations, each plasmid was injected bilaterally into the quadriceps femoris muscle of the mice (50 μL in each quadriceps), followed by local electroporation using an ECM830 square wave electroporation system (BTX Harvard Apparatus Company, Holliston, MA, USA). Each of the two-needle array electrodes delivered 15 pulses of 72 V (a 20-ms pulse duration at 200-ms intervals). For IN immunizations, each plasmid was administered into both nostrils (50 μL in each nostril) and mice were monitored in the upright position until complete recovery and vaccine absorption were assured. The mice were sacrificed 6 weeks and 10 weeks after treatment initiation. The spleens and left lungs were harvested and processed into single-cell suspensions. The right lungs were homogenized using glass homogenizers. Serial tenfold dilutions of lung homogenates in PBS were plated on 7H11 selective agar (BD) at the indicated time points. Plates were incubated at 37° C. and colony-forming units (CFU) were counted 4 weeks later.

### Immunogenicity Studies in Mice

Male and female C57BL/6 mice (8-10-week-old, Charles River Laboratory) were randomized to receive the rel_{Mtb} or the fusion DNA vaccine by the IM (20 or 200 μg) or IN (200 μg) route. The mice were sacrificed 6 weeks after the primary vaccination. Spleens, draining LNs, lungs and PBMCs were collected and processed into single-cell suspensions.

### Intracellular Cytokine Staining, Flow Cytometry Analysis and FluoroSpot

All the single-cell suspensions from spleens, draining LNs, lungs and PBMCs were stimulated individually with purified recombinant Rel_{Mtb} protein at 37° C. for various time intervals, from 12 hrs (IFN-γ, IL-17α, IL-2) to 24 hrs (TNF-α), depending on the cytokine of interest. For Intracellular Cytokine Staining (ICS), GolgiPlug cocktail (BD Pharmingen, San Diego, CA) was added for an additional 4 hours after stimulation (total, 16 and 28 hrs, respectively) and cells were collected using FACS buffer (PBS+0.5% Bovine serum albumin (Sigma-Aldrich, St. Louis, MO), stained with Zombie NIR™ Fixable Viability Kit (Biolegend Cat. No.: 423105) for 30 min, washed with PBS buffer, surface proteins were stained for 20 min, cells were fixed and permeabilized with buffers from Biolegend intracellular fixation/permeabilization set following manufacturer protocols (Cat. No. 421002), intracellular proteins were stained for 20 min, and samples were washed and resuspended with FACS buffer. The following anti-mouse mAbs were used for ICS: PercPCy5.5 conjugated anti-CD3 (Biolegend Cat. No 100217), FITC conjugated anti-CD4 (Biolegend Cat. No 100405), Alexa700 conjugated anti-CD8 (Biolegend Cat. No. 155022), PECy7 conjugated anti-TNF-α, (Biolegend Cat. No. 506323), APC conjugated anti-IFN-γ, (Biolegend Cat. No. 505809), BV421 conjugated anti-IL-2, (Biolegend Cat. No 503825), PE conjugated anti-IL-17α (Biolegend Cat. No 506903). The Attune™ NxT (Thermo Fisher Scientific, Waltham, MA), and a BD™ LSRII flow cytometer was used. Flow data were analyzed by FlowJo Software (FlowJo 10.8.1, LLC Ashland, OR). Flow analysis included alive, gated, total T lymphocytes, including CD4+T- and CD8+ T-cell subpopulations. For simplicity, the CD8+ subpopulation analysis is not reported if no substantial population to allow comparisons was detected (e.g., IL-17α). For FluoroSpot assays, kits with pre-coated plates for enumeration of cells secreting IFN-γ and IL-17A were purchased from Mabtech (Cat. No. FSP-414443-2). Spots were enumerated on an AID iSpot EliSpot/FluoroSpot Reader.

### Statistics

Pairwise comparisons of group mean values for log₁₀ CFU (microbiology data) and flow cytometry data were made by using one-way analysis of variance followed by Tukey's multiple comparisons test. Prism 9.3 (GraphPad Software, Inc. San Diego, CA) was utilized for statistical analyses and figure creation. For correlation of two nominal predictor variables to a continuous outcome variable, two-way analysis of variance followed by Tukey's multiple comparisons test was utilized. To illustrate the aggregate cytokine data per group (FIG. 4), Fraction of total analysis was used and is displayed in stacked bars and pie charts. In FIG. 4, cytokine data were normalized (the total sum of all the experimental groups=100%, total absolute number of alive cells=30,000). All error bars represent the estimation of the standard error of the mean, and all midlines represent the group mean. A significance level of α≤0.05 was set for all experiments.

### Example 2: Additional Investigation of the Mechanism of the Adjunctive Therapeutic Efficacy of the Intranasal MIP-3a/Rel_{Mtb} Vaccine

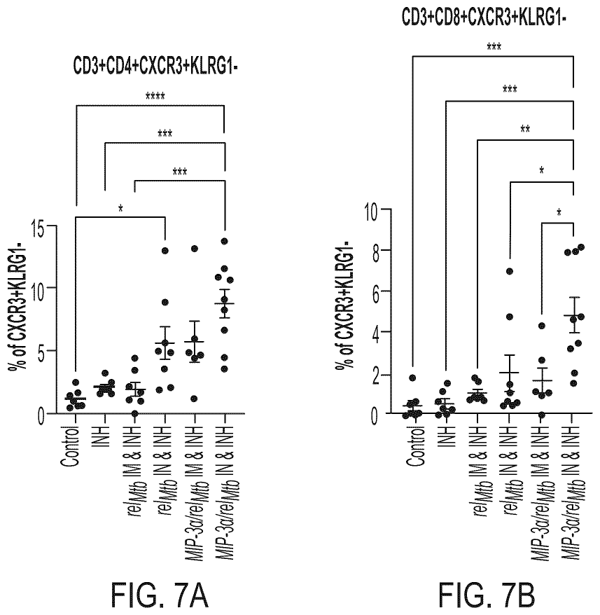

Continuing the work on investigating the mechanism of the adjunctive therapeutic efficacy of the intranasal (IN) MIP-3α/rel_{Mtb} (“fusion”) vaccine in Mtb-infected mice treated with isoniazid (INH), we evaluated T-cell lung homing markers in murine Mtb-infected lungs through flow cytometry. We found that the % of Rel_{Mtb}-specific CXCR3+KLRG-1−CD4+ or CD8+ T cells, a signature which is associated with protection against Mtb, is significantly higher in the IN fusion vaccination group compared to any other group (FIG. 7).

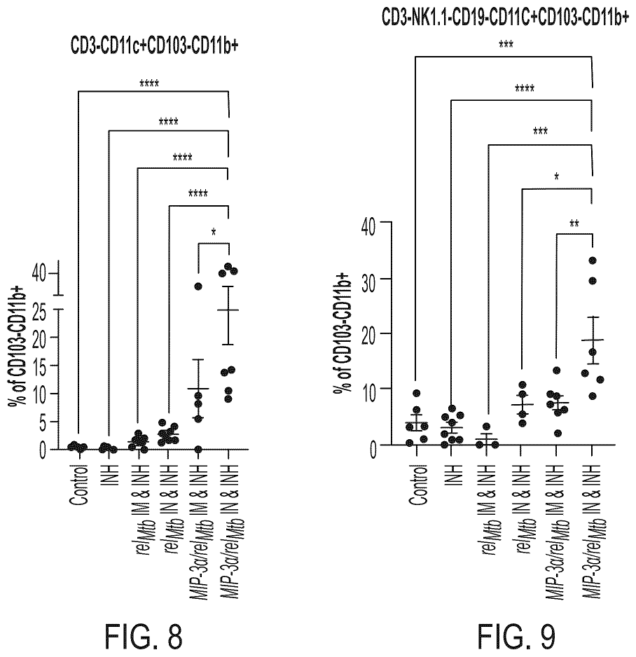

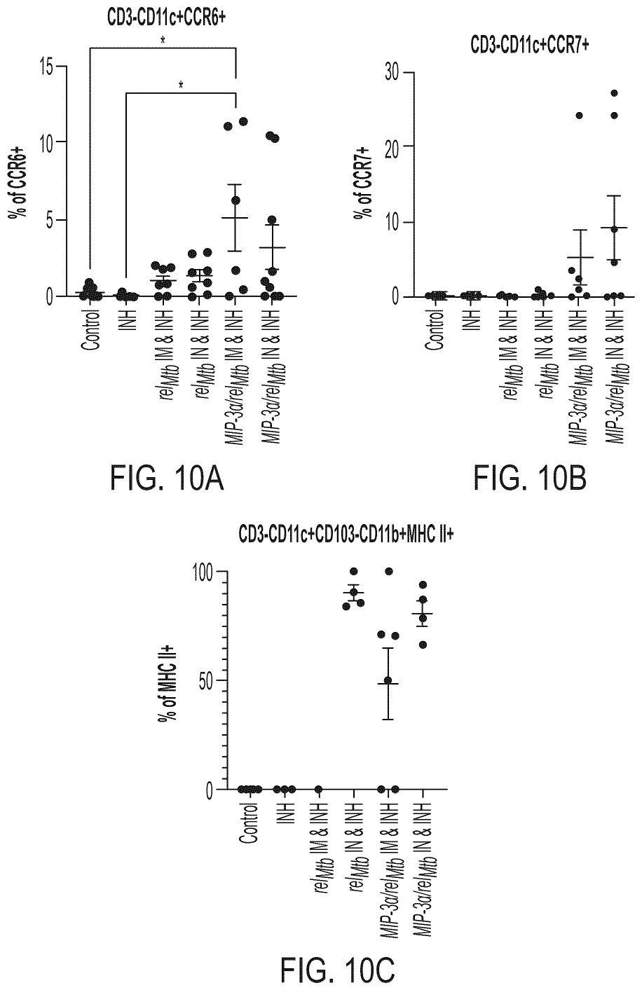

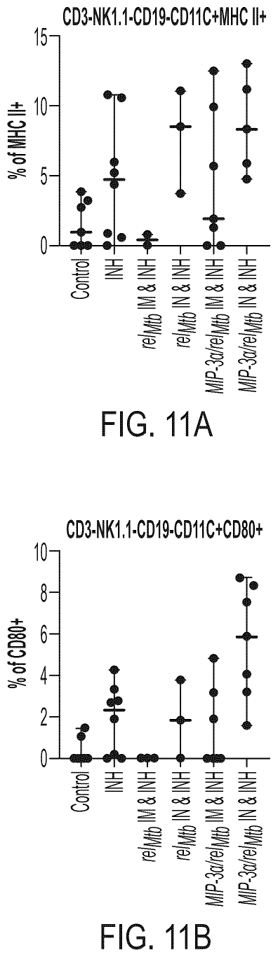

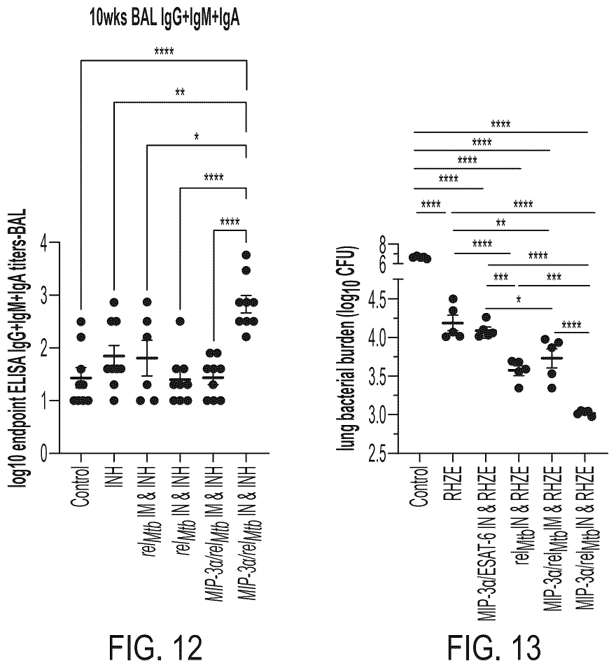

Since MIP-3α is a chemokine that targets the antigen to immature dendritic cells (DCs), we explored the dendritic cell infiltration of the murine lungs between the fusion and non-fusion vaccination groups, IN or intramuscularly (IM) administered, 6 and 10 weeks post prime vaccination and INH treatment initiation. We did not find significant differences in the numbers of the recruited classical dendritic cells in the lung parenchyma of the Mtb-infected mice across the groups in either time point. However, at six weeks upon treatment initiation, we found that the IN fusion vaccination group has significantly higher percentage of classical DCs type 2 compared to any other vaccination group (FIG. 8). At the same time point, we appreciated a positive trend of a higher number of the classical DCs type 2 in the IM fusion vaccination group compared to the IM rel_{Mtb} (“non-fusion”) vaccination group. At 10 weeks upon initiation of treatment, we optimized further our flow gating strategy, making that more selective for DCs, confirming the previous observation that the IN fusion vaccination group yielded the highest percentage of classical DCs type 2 (FIG. 9). Since classical DCs type 2 are associated with promoting the differentiation of naïve CD4+ T cells, it is suggested that the fusion vaccine leads to more effective differentiation of T cells. At the same time points we also assessed various DC activation/maturation markers namely CCR6+, CCR7+, MHCII+ and CD80+ in the murine lungs finding some encouraging trends favoring mainly the IN fusion vaccine (FIGS. 4 and 5). Although we know that the CCR6+ is the receptor of the MIP-3″ on the DC surface, and we hypothesized that it would be predominantly elevated in the fusion groups, the % of CCR6 expression did not reach statistical significance likely due to the fact the expression of CCR6 is a dynamic process through antigen presentation and 6 or 10 weeks upon the initiation of treatment might be too late as time points to depict the CCR6 upregulation since the T cell responses have been already observed (published data).

In the same experimental setting, we also checked Rel_{Mtb}-specific antibody responses through ELISA. Interestingly, we found that the IgA+IgM+IgG total antibody responses in the bronchoalveolar lavage collected from Mtb-infected mice were significantly increased in the IN fusion vaccination group at 10 weeks upon treatment initiation (FIG. 15).

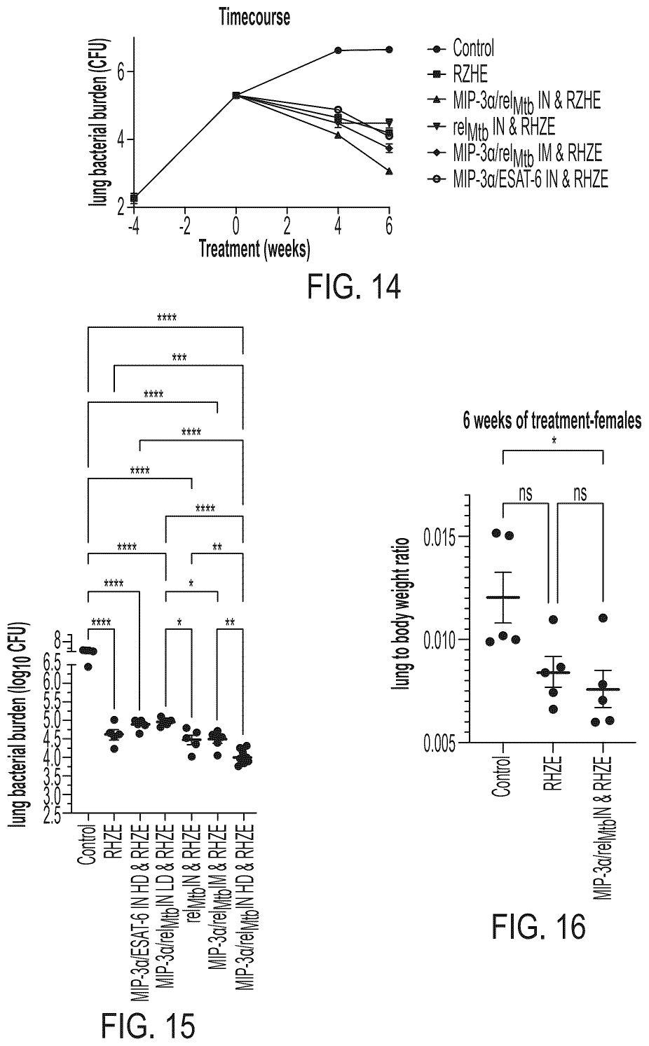

Next, we performed a new Mtb challenge animal experiment to assess the adjunctive therapeutic of the IN fusion vaccine to the full TB standard treatment regimen consisting of Rifampin (R), Isoniazid (H), Pyrazinamide (Z) and Ethambutol (E). Co-administration of the IN fusion vaccine along with the RHZE led to the greatest lung mycobactericidal reduction faster, in 6 weeks upon initiation of treatment, compared to RHZE alone (absolute reduction in lung bacillary load of 1.2 log 10), IN non-fusion vaccine (absolute reduction in lung bacillary load of 0.6 log 10), or IM fusion vaccine (absolute reduction in lung bacillary load of 0.7 log 10) (FIGS. 13 and 14). Both IN non-fusion and IM fusion vaccine combined with RHZE offered significantly greater lung mycobacterial burden reduction compared to RHZE alone. Of note, we also included in the comparator vaccination groups, the IN MIP-3α/ESAT-6 vaccination group. The esxA gene (the gene encoding the protein ESAT-6) was fused to the mouse MIP3-α gene. In contrast to Rel_{Mtb}, ESAT-6 is a non-stringent response Mtb antigen and conventionally viewed as one of the immunodominant Mtb antigens. The fusion product MIP-3α/ESAT-6 was cloned into pSectag2B. Proper insertion was confirmed by sequencing and the expression of target genes was confirmed by transfection of 293T cells in lysates and supernatants. Vaccination plasmids were selected by ampicillin and extracted from E. coli DH5-a. The dose, administration scheme (3 weekly doses) and route of the MIP-3α/ESAT-6 vaccine was the same with our optimized vaccination strategy (IN fusion vaccine or IN MIP-3α/rel_{Mtb}) in an effort to allow for direct comparison in the adjunctive therapeutic efficacy of the respective vaccination strategies. IN MIP-3α/ESAT-6 vaccine did not offer any adjunctive therapeutic efficacy to RHZE alone. This finding strengthens further our initial hypothesis that rel_{Mtb} is a promising host-directed therapeutic target against TB which continues to be presented during TB treatment. We also tested a 10× lower dose of the IN fusion vaccine (20 μg) co-administered with RHZE; the vaccination group receiving this dose did not have significant difference than the RHZE alone group implying that there is a dose dependent effect (FIG. 9). In terms of the animal lung weights, which is usually a surrogate of lung inflammation, the normalized lung weights from the IN fusion vaccination group were significantly lower compared to the control in the end of treatment (6 weeks), but did not reach significance compared to the RHZE group alone (FIG. 16). The latter finding is not surprising since the lung weight reduction lags usually behind compared to the lung mycobacterial reduction.

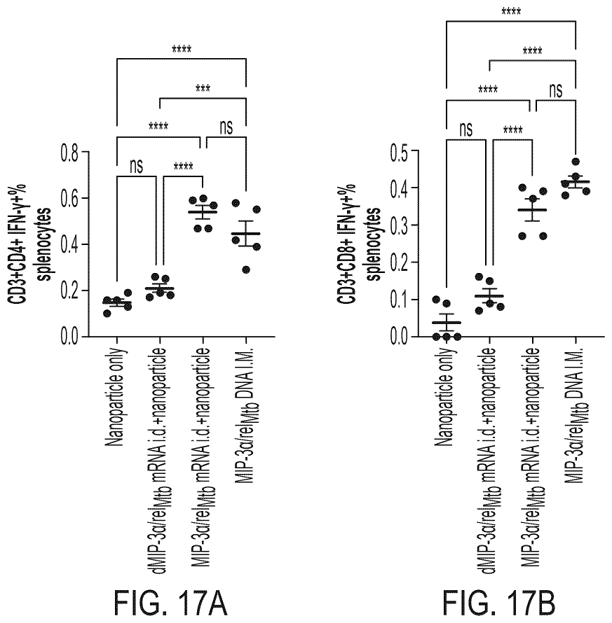

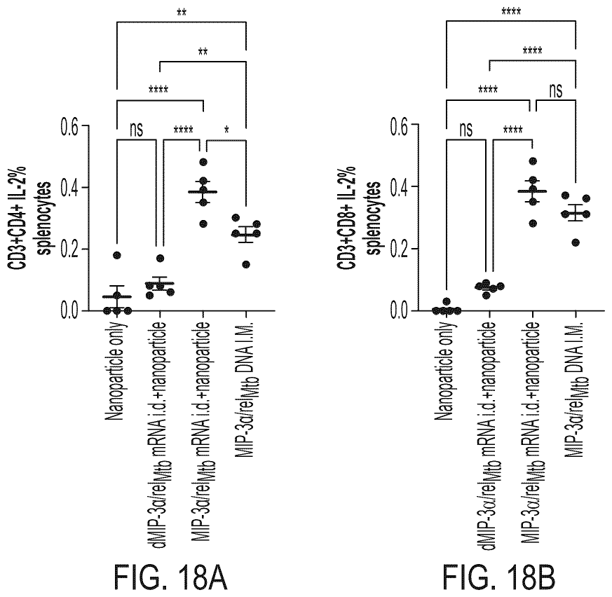

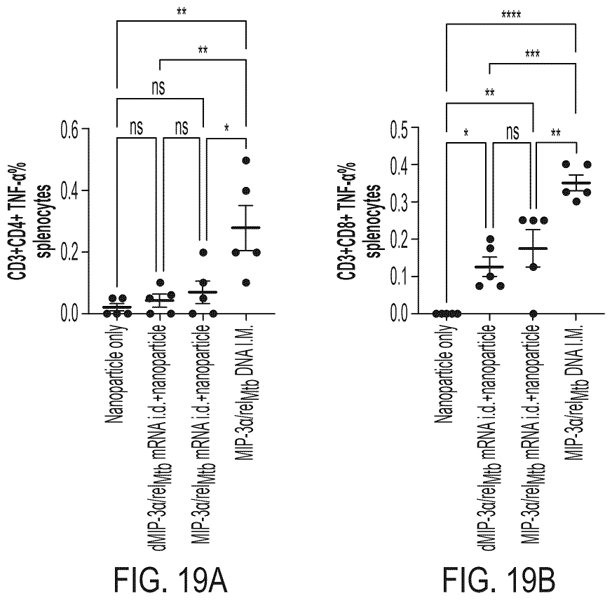

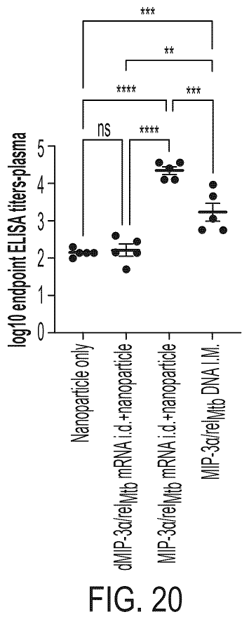

Last, we have started working on the transition of this effective DNA therapeutic TB vaccine to the mRNA platform which can be easily and safely applied to humans. We have performed a non-challenge animal experiment to compare the immunogenicity of an mRNA vaccine encapsulated with biodegradable polymer nanoparticles (NPs) expressing MIP-3α/rel_{Mtb} with our optimized DNA fusion vaccine expressing MIP-3α/rel_{Mtb}. NPs have been shown to be an efficient carrier to deliver mRNA in vivo, which allows rapid uptake and expression in host cells and finally leads to robust adaptive humoral and cellular immune responses. Since our collaborators working with NPs have previous extensive experience with the intradermal (i.d.) administration of nanoparticle-encapsulated mRNAs, we initially compared the immune responses elicited by an i.d. mRNA vaccine expressing MIP-3α/rel_{Mtb} (6 μg) with the ones elicited by the established IM fusion DNA vaccine (20 μg) (the latter followed by electroporation). Both vaccines elicited similar numbers of Rel_{Mtb}-specific CD4+ and CD8+ T cells producing-IFN-γ as assessed by intracellular staining (FIG. 17). Interestingly, the i.d. mRNA vaccine expressing MIP-3α/rel_{Mtb} elicited stronger IL-2 producing CD4+ T cells than the IM fusion DNA vaccine (FIG. 18) while the opposite was observed with the TNF-α producing CD4+ and CD8+ T cells (FIG. 19). Also, the group receiving the i.d. mRNA vaccine expressing MIP-3α/rel_{Mtb} elicited higher rel_{Mtb}-specific antibody titers than the IM fusion DNA vaccine (FIG. 20). Working with our collaborators, we next performed an In Vivo Imaging experiment (IVIS) in an effort to determine the appropriate dose of the IN NP-encapsulated mRNA fusion vaccine to correspond with the IN DNA fusion vaccine efficacious dose. 5, 10 and 20 μg of mRNA firefly luciferase administration showed that higher doses may increase the nose and lung transfection. Then, in an effort to assess the antigen-specific immune responses per dose with an IN administered mRNA vaccine but also to preliminary assess for safety, we proceeded to an IN ovalbumin (OVA) mRNA vaccination experiment. We administered 5, 10 or 20 μg OVA mRNA per mouse weekly for three weeks and spleens, lungs, mediastinal lymph nodes, peripheral blood mononuclear cells (PBMCs), plasma and bronchoalveolar lavage (BAL) were collected at 6 weeks upon initiation of treatment. Toxicity was documented in the group receiving the dose of 20 μg OVA mRNA leading to significant animal loss. This animal group was eliminated. 5 and 10 μg of the OVA mRNA vaccine elicited similar OVA-specific IFN-γ, IL-2, TNF-α and IL-17A-producing T cells in single cell suspensions from spleens, lungs, mediastinal LNs and PBMCs, as assessed by intracellular staining. IgG antibody responses in plasma and BAL were also similar between the two groups as assessed by ELISA. We are currently running an Mtb-challenge animal experiment to compare the adjunctive therapeutic efficacy of the IN fusion mRNA vaccine (5 and 10 μg) encapsulated with NPs with our optimized IN DNA fusion vaccine when co-administered with RHZE. Results to follow.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nucleic acid vaccine composition comprising a synthetic polynucleotide encoding a Mycobacterium tuberculosis (Mtb) RelA-SpoT homolog (RSH) protein, Rel_{Mtb}, or a functional portion, fragment, or variant thereof, conjugated to a macrophage inflammatory protein-3 alpha (MIP-3α) or other chemokine that binds to a chemokine receptor 6 (CCR6), or a functional portion, fragment, or variant thereof, or to an antibody, or antigen binding portion thereof, that binds to a CCR6.

2. The nucleic acid vaccine composition of claim 1, wherein the synthetic polynucleotide comprises DNA.

3. The nucleic acid vaccine composition of claim 1, wherein the synthetic polynucleotide comprises RNA.

4. The nucleic acid vaccine composition of claim 1, wherein the synthetic polynucleotide comprises the nucleotide sequence of SEQ ID. NOS: 1 and 3.

5. The nucleic acid vaccine composition of claim 4, wherein the synthetic polynucleotide further comprises the nucleotide sequence of SEQ ID. NO: 2.

6. The nucleic acid vaccine composition of claim 1, wherein the MIP-3α is murine or human.

7. The nucleic acid vaccine composition of claim 1, wherein the synthetic polynucleotide is codon-optimized for expression in a mammalian cell.

8. The nucleic acid vaccine composition of claim 7, wherein the mammalian cell is a human cell.

9. A recombinant nucleic acid vector encoding the nucleic acid vaccine compositions of claim 1.

10. The vector of claim 9, wherein the vector is a pSectag2B plasmid or a pVax1 plasmid.

11. A pharmaceutical composition comprising the recombinant nucleic acid vector of claim 9 and a pharmaceutically acceptable carrier.

12. The pharmaceutical composition of claim 11, further comprising at least one additional biologically active agent.

13. The pharmaceutical composition of claim 11, wherein the pharmaceutically acceptable carrier comprises a lipid nanoparticle (LNP), a polymeric nanoparticle, a lipidoid, a liposome, a lipoplex, a peptide carrier, a nanoparticle mimic, or a conjugate thereof.

14. A method of providing prophylaxis to, and/or treating an Mtb infection in, a subject in need thereof comprising administering to the subject an effective amount of the composition of claim 9.

15. The method of claim 14, wherein the composition is administered to the subject prior to, concurrent with, and/or after administering at least one antibiotic agent to the subject.

16. The method of claim 14, wherein the composition is administered as one or more boost doses after an initial administration of the composition to the subject.

17. The method of claim 14, wherein the composition is administered intramuscularly and/or intranasally to the subject.

18. A vaccine composition, comprising a polypeptide that comprises a Mycobacterium tuberculosis (Mtb) RelA-SpoT homolog (RSH) protein, Rel_{Mtb}, or a functional portion, fragment, or variant thereof, conjugated to a macrophage inflammatory protein-3 alpha (MIP-3α) or other chemokine that binds to a chemokine receptor 6 (CCR6), or a functional portion, fragment, or variant thereof, or to an antibody, or antigen binding portion thereof, that binds to a CCR6.

19. A pharmaceutical composition comprising the vaccine composition of claim 18, and a pharmaceutically acceptable carrier.

20. The pharmaceutical composition of claim 19, further comprising at least one additional biologically active agent.

21. The vaccine composition of claim 18, wherein the polypeptide comprises the amino acid sequence of SEQ ID. NOS: 5 and 7.

22. The vaccine composition of claim 21, wherein the polypeptide further comprises the amino acid sequence of SEQ ID. NO: 6.

23. A method of providing prophylaxis to, and/or treating an Mtb infection in a subject in need thereof comprising administering to the subject an effective amount of the composition of claim 18.

24. The method of claim 23, wherein the composition is administered to the subject prior to, concurrent with, and/or after administering at least one antibiotic agent to the subject.

25. The method of claim 23, wherein the composition is administered as one or more boost doses after an initial administration of the composition to the subject.

26. The method of claim 23, wherein the composition is administered intramuscularly and/or intranasally to the subject.
